(19) United States
(12) Patent Application Publication
(10) Pub. No.: US 2020/0300866 A1
(43) Pub. Date: Sep. 24, 2020

(54) DIAGNOSIS OF CROHN'S DlSEASE AND ULCERATIVE COLITIS

(71) Applicant: Flora Innovation AB, Göteborg (SE)

(72) Inventors: Sofia ÖSTMAN, Sävedalen (SE); Agnes WOLD, Gothenburg (SE); Robert SAALMAN, Mölndal (SE)

(21) Appl. No.: 16/754,772

(22) PCT Filed: Oct. 9, 2018
(86) PCT No.: PCT/SE2018/051037

§ 371 Date: Apr. 9, 2020

(30) Foreign Application Priority Data
Oct. 9, 2017 (SE) 1751246-8

(51) Int. Cl. G01N 33/68; C07K 14/705; G01N 33/569; G01N 33/72

(52) U.S. Cl. CPC G01N 33/6893; C07K 14/70578; G01N 33/56972; G01N 2800/065; G01N 33/721; G01N 2333/70539; G01N 2333/70578; G01N 33/56977

(57) ABSTRACT

The present invention relates to a method for determining if a subject suffers from Crohn's disease or from ulcerative colitis, wherein the method comprises the steps of determining if the subject suffers from inflammatory bowel disease and determining proportion of B-cells which are CD27+ in a sample from the subject. The method may further comprise determining the proportion of CD8+ T-cells which are HLA-DR+ in a sample from the subject. The present invention also relates to biomarkers for use as a marker in the diagnosis of inflammatory bowel disease, Crohn's disease ulcerative colitis. The present invention also relates to a kit for the diagnosis of Crohn's disease and/or ulcerative colitis.

### TECHNICAL FIELD OF THE INVENTION

This invention relates to a method for diagnosing Crohn's disease and ulcerative colitis.

### BACKGROUND

Inflammatory bowel disease (IBD) is a chronic, systemic, inflammatory condition that mainly affects the gastrointestinal tract, although it is often also associated with extraintestinal manifestations and autoimmune disorders. While the etiology of IBD remains unclear, it is thought to involve a complex interplay between genetic predisposition, gut microbiota composition, and dysregulated immune responses.

It is important to distinguish IBD from the less severe condition irritable bowel syndrome (IBS), since they involve very different treatments. The symptoms (i.e. abdominal pain, constipation, and urgent bowel movements) of IBD and IBS overlap, but IBS is a non-inflammatory condition, while inflammatory bowel disease IBD causes chronic inflammation of the intestinal tract, which may lead to ulcers, scar tissue, and even permanent damage of the intestinal tract. Increased levels of faecal calprotectin may be used as a diagnostic marker for IBD.

IBD encompasses the two major subcategories Crohn's disease and ulcerative colitis. Both of these subtypes are characterized by the presence of severe inflammation in the gut wall. In ulcerative colitis, the intestinal inflammatory infiltrate is mainly restricted to the large intestine (the colon and rectum), while in Crohn's disease, it may occur at any site along the gastrointestinal tract (from mouth to anus) and often involves the entire gut wall.

Some of the medications available for treatment can be used for either ulcerative colitis or Crohn's disease, however, some medications are used for only Crohn's disease or only ulcerative colitis. The approach to surgical treatment also differs between the two conditions. Thus, it is important to determine if a subject is suffering from Crohn's disease or from ulcerative colitis. Today, diagnosis is made based on the Porto criteria which include evaluation of the clinical picture, radiologic imaging techniques, such as X-rays, CT scans and MRIs, and gastrointestinal endoscopy findings. These diagnostic tools are expensive as well as time consuming and are also perceived as uncomfortable by most patients. In a substantial number of patients it is still difficult to distinguish between Crohn's disease and ulcerative colitis. Thus, there is a need of additive diagnostic tools which facilitate this diagnostic process.

An object of the present invention is to introduce an additive tool to discriminate between Crohn's disease and ulcerative colitis.

### SUMMARY OF THE INVENTION

According to a first aspect of the invention, the above and other objects of the invention are achieved, in full or at least in part, by a method as defined by claim 1. According to this claim the above object is achieved by a method for determining if a subject suffers from Crohn's disease or from ulcerative colitis, wherein the method comprises the steps of determining if the subject suffers from inflammatory bowel disease; determining the proportion of B-cells which are CD27⁺ in a sample S1 from the subject; and comparing said proportion with a threshold value TV_{CD27}. If said subject suffers from inflammatory bowel disease, a proportion lower than said threshold value TV_{CD27} is indicative of that said subject suffers from Crohn's disease. If said subject suffers from inflammatory bowel disease, a proportion higher than said threshold value TV_{CD27} is indicative of that said subject suffers from ulcerative colitis.

One advantage of this method for determining if a subject suffers from Crohn's disease or from ulcerative colitis is that the diagnosis can be determined from a blood sample. Thus, in a substantial number of patients this may minimize the need of repeated endoscopic procedures for diagnosing Crohn's disease. Expensive, time consuming and uncomfortable methods may therefore be reduced.

The subject may be a child.

The subject may be an adolescent.

The subject may be an adult.

The subject may not have received treatment for symptoms associated with IBD.

The subject may have received treatment for symptoms associated with IBD.

The proportion of B-cells which are CD27⁺ may be expressed as percentage (%) of the total number of B-cells in a sample.

Instead of determining the proportion of B-cells which are CD27⁺, the total number of B-cells which are CD27⁺ in a predetermined volume of a sample may be determined.

The sample S1 may be a blood sample.

The sample S1 may be a tissue sample, such as a biopsy from the intestinal tract, such as from the colon, rectum, ileum, or duodenum

The threshold value TV_{CD27} may be determined as described below.

The threshold value TV_{CD27} may be different for different groups of subjects, such as for children, adolescents, adults, subjects which have been suffering from symptoms associated with IBD for a short time period, subjects which have been suffering from symptoms associated with IBD for a long time period, subjects which have not received treatment for symptoms associated with IBD or subjects which have received treatment for symptoms associated with IBD.

According to one embodiment, the threshold value TV_{CD27} may be between 5% and 30%, such as between 10% and 20%, such as between 13% and 18%, such as between 15% and 17%. Examples of TV_{CD27} are 12%, 14%, 16.5%, 18%, 20% and 22%.

According to one embodiment, the TV_{CD27} is 16.5%.

If the total number of B-cells which are CD27⁺ in a predetermined volume of a sample is used, the threshold value may be between 2.5×10⁴ cells/mL blood and 8×10⁴ cells/mL blood, such as between 3×10⁴ cells/mL blood and 7×10⁴ cells/mL blood, such as between 4×10⁴ cells/mL blood and 6.5×10⁴ cells/mL blood, such as between 4.6×10⁴ cells/mL blood and 5.8×10⁴ cells/mL blood.

Examples of threshold values for number of B-cells which are CD27⁺ are 3×10⁴ cells/mL blood, 4×10⁴ cells/mL blood, 4.5×10⁴ cells/mL blood, 5×10⁴ cells/mL blood, 5.5×10⁴ cells/mL blood, 6×10⁴ cells/mL blood.

According to another embodiment, the method further comprises the step of determining the proportion of B-cells which are CD23⁺ in a sample S2 from the subject; calculating the ratio of the proportions of CD23⁺ B-cells and CD27⁺ B-cells; and comparing said ratio with a threshold value TV_{CD23/CD27}. If said subject suffers from inflammatory bowel disease, a ratio higher than said threshold value TV_{CD23/CD27} is indicative of that said subject suffers from Crohn's disease. If said subject suffers from inflammatory bowel disease, a ratio lower than said threshold value TV_{CD23/CD27} is indicative of that said subject suffers from ulcerative colitis.

One advantage of this embodiment is that the diagnosis can be determined with higher precision.

The proportion of B-cells which are CD23⁺ may be expressed as percentage (%) of the total number of B-cells in a sample.

Instead of determining the proportion of B-cells which are CD23⁺, the total number of B-cells which are CD23⁺ in a predetermined volume of a sample may be determined. In this case the total number of B-cells which are CD27⁺ in a predetermined volume of a sample is used to calculate the ratio.

The sample S2 may be a blood sample.

The sample S2 may be a tissue sample, such as a biopsy from the intestinal tract, such as from the colon, rectum, ileum, or duodenum.

The threshold value TV_{CD23/CD27} may be determined as described below.

According to one embodiment, the threshold value TV_{CD23/CD27} may be between 0.5 and 8, such as between 2 and 7, such as between 3 and 6, such as between 4 and 5. Examples of TV_{CD23/CD27} are 1, 2, 3, 4, 5, 6, and 7.

According to one embodiment, the TV_{CD23/CD27} is 5.

According to one embodiment each of the samples S1 and S2 independently is a blood sample or a tissue sample.

The blood sample may be supplemented with a substance which prevents coagulation and/or aggregation of the blood cells. One example of such a substance is EDTA. Other examples are hirudin, citrate, and heparin.

The tissue sample may be a biopsy from the intestinal tract, such as from the colon, rectum, ileum, or duodenum.

According to yet another embodiment, the step of determining that the subject suffers from IBD comprises determining the amount of hemoglobin in a sample S3 and the amount of orosomucoid in a sample S4 from the subject; calculating the ratio of the amount of hemoglobin in the sample S3 and the amount of orosomucoid in the sample S4; and comparing said ratio with a threshold value TV_{H/O}. A ratio lower than said threshold value TV_{H/O} is indicative of that said subject suffers from IBD.

One advantage of this method is that the diagnosis may be determined from a blood sample and a plasma sample. Thus, in a substantial number of patients this may minimize the need of repeated endoscopic procedures for diagnosing Crohn's disease. Expensive, time consuming and uncomfortable methods may therefore be reduced.

Another advantage is that the ratio of hemoglobin and orosomucoid provides a reliable tool to distinguish subjects suffering from IBD from subjects with similar symptoms, but which do not have IBD.

Yet another advantage of this method is that the diagnosis can be determined with higher precision.

The sample S3 may be a blood sample.

The sample S4 may be plasma.

The sample S4 may be serum.

Hemoglobin may be measured as g/L blood.

Orosomucoid may be measured as g/L blood.

In one embodiment, hemoglobin is measured as g/L blood and orosomucoid is measured as g/L blood.

The threshold value TV_{H/O} may be determined as described below.

According to one embodiment, the threshold value TV_{H/O} is between 100 and 200, such as between 125 and 190, such as between 140 and 180, such as between 150 and 170. Examples of TV_{H/O} are 120, 130, 140, 150, 160, 168, 170, and 180.

According to one embodiment, the TV_{H/O} is 168.

According to another embodiment, the step of determining that the subject suffers from inflammatory bowel disease comprises determining the amount of hemoglobin in a sample S3 and calprotectin in a sample S5 from the subject; calculating the ratio of the amounts of hemoglobin in the sample S3 and calprotectin in the sample S5; and comparing said ratio with a threshold value TV_{H/C}. A ratio lower than said threshold value TV_{H/C} is indicative of that said subject suffers from inflammatory bowel disease.

The sample S3 may be a blood sample.

The sample S5 may be faeces.

Hemoglobin may be measured as g/L blood.

Calprotectin may be measured as mg/kg faeces.

In one embodiment, hemoglobin is measured as g/L blood and calprotectin is measured as mg/kg faeces.

The threshold value TV_{H/C} may be determined as described below.

According to one embodiment, the threshold value TV_{H/C} is between 0.1 and 5, such as between 0.2 and 4, such as between 0.4 and 3, such as between 0.6 and 2, such as between 0.8 and 1. Examples of TV_{H/C} are 0.4, 0.6, 0.8, 0.94, 1, 2, 3, and 4.

According to one embodiment the TV_{H/C} is 0.94.

According to yet another embodiment, the step of determining that the subject suffers from inflammatory bowel disease comprises the steps of determining the amount of orosomucoid in a sample S4 from the subject; and comparing said amount of orosomucoid with a threshold value for orosomucoid TV_{O}. An amount of orosomucoid higher than said threshold value TV_{O} is indicative of that said subject suffers from IBD.

The sample S4 may be plasma.

The sample S4 may be serum.

Orosomucoid may be measured as g/L blood.

In one embodiment, orosomucoid is measured as g/L blood.

The threshold value TV_{O} may be determined as described below.

According to one embodiment, the threshold value TV_{O} is between 0.5 and 1 g/L blood, such as between 0.6 and 0.9 g/L blood, such as between 0.65 and 0.85 g/L blood, such as between 0.7 and 0.8 g/L blood. Examples of TV_{O} are 0.5 g/L blood, 0.6 g/L blood, 0.7 g/L blood, 0.74 g/L blood, 0.8 g/L blood, 0.9 g/L blood and 1 g/L blood.

According to one embodiment, the TV_{O} is 0.74 g/L blood.

According to one embodiment, the step of determining that the subject suffers from IBD comprises the steps of determining the amount of calprotectin in a sample S5 from the subject; and comparing said amount of calprotectin with a threshold value TV_{C} for calprotectin. An amount of calprotectin higher than said threshold value TV_{C} is indicative of that said subject suffers from IBD.

The sample S5 may be faeces.

Calprotectin may be measured as mg/kg faeces.

In one embodiment, calprotectin is measured as mg/kg faeces.

The threshold value TV_{C} may be determined as described below.

According to one embodiment, the threshold value TV_{C} is between 50 and 300 mg/kg faeces, such as between 75 and 250 mg/kg faeces, such as between 100 and 200 mg/kg faeces, such as between 130 and 175 mg/kg faeces, such as between 140 and 170 mg/kg faeces. Examples of TV_{C} are 75 mg/kg faeces, 100 mg/kg faeces, 120 mg/kg faeces, 140 mg/kg faeces, 150 mg/kg faeces, 160 mg/kg faeces, 166.5 mg/kg faeces, 170 mg/kg faeces, 180 mg/kg faeces, 200 mg/kg faeces, and 220 mg/kg faeces.

According to one embodiment, the TV_{C} is 166.5 mg/kg faeces.

According to another embodiment, the method further comprises the steps of determining the proportion of CD8⁺ T-cells which are HLA-DR⁺ in a sample S6 from the subject; and comparing said proportion with a threshold value TV_{HLA-DR}. A proportion of CD8⁺ T-cells which are HLA-DR⁺ that is higher than said threshold value TV_{HLA-DR} is indicative of that said subject suffers from ulcerative colitis.

The proportion of CD8⁺ T-cells which are HLA-DR⁺ may be expressed as percentage (%) of cells in a sample.

Instead of determining the proportion of CD8⁺ T-cells which are HLA-DR⁺, the total number of CD8⁺ T-cells which are HLA-DR⁺ in a predetermined volume of a sample may be determined.

The sample S6 may be blood.

The sample S6 may be a tissue sample, such as a biopsy from the intestinal tract, such as from the colon, rectum, ileum, or duodenum.

The threshold value TV_{HLA-DR} may be determined as described below.

According to one embodiment, the threshold value TV_{HLA-DR} for the proportion of CD8⁺ T-cells which are HLA-DR⁺ is between 10 and 40%, such as between 15 and 30%, such as between 20 and 28%, such as between 22 and 26%. Examples of TV_{HLA-DR} for CD8⁺ T-cells are 12%, 15%, 17%, 20%, 22%, 25.5%, 27%, 30% and 35%.

The threshold value TV_{HLA-DR} for the proportion of CD8⁺ T-cells which are HLA-DR⁺ may also denoted TV_{HLA-DR(CD8+)P}.

According to one embodiment, the TV_{HLA-DR} for CD8⁺ T-cells is 25.5%.

If the total number of CD8⁺ T-cells which are HLA-DR⁺ in a predetermined volume of a sample is used, the threshold value typically is between 0.5×10⁵ and 3×10⁵ cells/mL blood, such as between 1×10⁵ and 2×10⁵ cells/mL blood, such as between 1.5×10⁵ and 1.8×10⁵ cells/mL blood. Examples of TV_{HLA-DR} for total number of CD8⁺ T-cells which are HLA-DR⁺ (also denoted TV_{HLA-DR(CD8+)N}) are 0.5×10⁵ cells/mL blood, 1×10⁵ cells/mL blood, 1.5×10⁵ cells/mL blood, 2×10⁵ cells/mL blood, and 2.5×10⁵ cells/mL blood.

In an alternative embodiment, in addition to the proportion of CD8⁺ T-cells which are HLA-DR⁺, the proportion or total number of CD8⁺ T-cells which are CD62L⁺ in a sample from the patient is also be determined. The sample may be blood. The ratio of the proportion of CD8⁺ T-cells which are HLA-DR⁺ and the proportion of CD8⁺ T-cells which are CD62L⁺ may be determined. Alternatively, the ratio is calculated as the ratio of the total number of CD8⁺ T-cells which are HLA-DR⁺ and the total number of CD8⁺T-cells which are CD62L⁺. A ratio higher than threshold value TV_{HLA-DR/CD}62L is indicative of that said subject suffers from ulcerative colitis. The threshold value TV_{HLA-DR/CD62L} may be between 0.2 and 0.8, such as between 0.3 and 0.7, such as between 0.4 and 0.6, such as between 0.45 and 0.5. Examples of TV_{HLA-DR/CD62L} are 0.3, 0.35, 0.4, 0.45, 0.48, 0.5, 0.6 and 0.7.

According to one embodiment, the TV_{HLA-DR/CD62L} is 0.48.

Alternatively, the proportion of other T cell subsets which are HLA-DR⁺ may be determined by using CD4 or the pan T cell marker CD3 in a sample S6 from the subject; and comparing said proportion with a threshold value TV_{HLA-DR} for CD3⁺ or CD4⁺ T-cells. A proportion of CD3⁺ T-cells which are HLA-DR⁺ higher than said threshold value TV_{HLA-DR} for CD3⁺ or CD4⁺ T-cells is indicative of that said subject suffers from ulcerative colitis.

Thus, according to another embodiment, the method further comprises the steps of determining the proportion of CD3⁺ T-cells which are HLA-DR⁺ in a sample S6 from the subject; and comparing said proportion with a threshold value TV_{HLA-DR} for CD3⁺ T cells; wherein, a proportion of CD3⁺ T-cells which are HLA-DR⁺ higher than said threshold value TV_{HLA-DR} for the proportion of CD3⁺ T-cells which are HLA-DR⁺ is indicative of that said subject suffers from ulcerative colitis.

The threshold value TV_{HLA-DR} for the proportion of CD3⁺ T-cells which are HLA-DR⁺ may also denoted TV_{HLA-DR(CD3+)P}.

According to another embodiment, the samples S1, S2, S3, S4, S5 and S6 are independently chosen from the group consisting of blood, plasma, serum, faeces, or tissue sample.

A blood sample may be supplemented with a substance which prevents coagulation and/or aggregation of the blood cells. One example of such a substance is EDTA. Other examples are hirudin, citrate, and heparin.

Some of the samples may be the same. Thus, one or more of the samples S1, S2, S3, S4 and S6 may be the same sample, such as a blood sample.

The threshold values TV_{CD27}, TV_{CD23/CD27}, TV_{H/O}, TV_{H/C}, TV_{O}, TV_{C} and TV_{HLA-DR} may be different for different groups of subjects, such as for children, adolescents, adults, subjects which have been suffering from symptoms associated with IBD for a short time period, subjects which have been suffering from symptoms associated with IBD for a long time period, subjects which have not received treatment for symptoms associated with IBD or subjects which have received treatment for symptoms associated with IBD.

According to a second aspect of the invention CD27⁺ B-cells for use as a marker in the diagnosis of Crohn's disease is also provided.

One advantage of CD27⁺ B-cells as a marker in the diagnosis of Crohn's disease is that this marker can be determined from a blood sample. Thus, in a substantial number of patients this may minimize the need of repeated endoscopic procedures for diagnosing Crohn's disease. Expensive, time consuming and uncomfortable methods may therefore be reduced.

According to a second aspect of the invention the ratio of CD23⁺ B-cells and CD27⁺ B-cells for use as markers in the diagnosis of Crohn's disease is also provided.

One advantage of the ratio of CD23⁺ B-cells and CD27⁺ B-cells as a marker in the diagnosis of Crohn's disease is that this marker can be determined from a blood sample.

Thus, in a substantial number of patients this may minimize the need of repeated endoscopic procedures for diagnosing Crohn's disease. Expensive, time consuming and uncomfortable methods may therefore be reduced.

According to a second aspect of the invention the ratio of hemoglobin and orosomucoid for use as a marker in the diagnosis of inflammatory bowel disease is provided.

One advantage of the ratio of hemoglobin and orosmucoid as a marker in the diagnosis of Crohn's disease is that this marker can be determined from a blood sample. Thus, the more expensive, time consuming and uncomfortable methods for diagnosing Crohn's disease may be avoided.

Another advantage is that the ratio of hemoglobin and orosmucoid provides a reliable tool to distinguish subjects suffering from IBD from subjects with similar symptoms, but which do not have IBD.

According to a third aspect of the invention the proportion of CD8⁺ T-cells which are HLA-DR⁺ for use as a marker in the diagnosis of ulcerative colitis is provided. The T cells which are HLA-DR⁺ can also be analyzed by using CD4 or the pan-T cell marker CD3.

One advantage of T-cells which are HLA-DR⁺ for use as a marker in the diagnosis of ulcerative colitis is that this marker can be determined from a blood sample. Thus, in a substantial number of patients this may minimize the need of repeated endoscopic procedures for diagnosing Crohn's disease. Expensive, time consuming and uncomfortable methods may therefore be reduced.

According to a fourth aspect, the proportion of CD3⁺ T-cells which are HLA-DR⁺ for use as a marker in the diagnosis of ulcerative colitis is also provided.

According to a fifth aspect, a kit for the diagnosis of Crohn's disease and/or ulcerative colitis comprising a B-cell marker; antibodies directed against CD27; and instructions for use is also provided.

The B-cell marker may be CD19.

The B-cell marker may be CD20.

The B-cell marker may be immunoglobulins, including IgD, IgM, IgG, IgA, and IgE.

According to one embodiment, the kit further comprises antibodies directed against CD23.

According to one embodiment, the kit further comprises a T-cell marker; antibodies directed against HLA-DR and optionally antibodies directed against CD62L.

The T-cell marker may be CD3.

The T-cell marker may be CD8.

The T-cell marker may be CD4.

According to one embodiment, the kit further comprises reagents to measure levels of orosomucoid.

According to a sixth aspect, a method of treatment of Crohn's disease in a subject is provided, wherein the method comprises performing the steps of the method for determining if a subject suffers from Crohn's disease or from ulcerative colitis according to the present invention, and, when the subject is found to suffer from Crohn's disease, further comprising the step of administering to said subject at least one medicament for treating Crohn's disease.

The medicament may be chosen from the group consisting of steroids, such as budesonide, prednisolone, and betamethasone, or immune modulators, such as azathioprine, mercaptopurine and methotrexate, or biological treatments such as, adalimumab and infliximab, or 5-aminosalicylates, such as sulfasalazine, mesalamine, balsalazide, and olsalazine, or antibiotics, such as metronidazole.

According to a seventh aspect, a method of treatment of ulcerative colitis in a subject is provided, wherein the method comprises performing the steps of the method for determining if a subject suffers from Crohn's disease or from ulcerative colitis according to the present invention, and, when the subject is found to suffer from ulcerative colitis, further comprising the step of administering to said subject at least one medicament for treating ulcerative colitis.

The medicament may be chosen from the group consisting of steroids, such as prednisolone and betamethasone, or 5-aminosalicylates, such as sulfasalazine, mesalamine, balsalazide, and olsalazine, or biological treatments such as, adalimumab and infliximab, or calcineurin inhibitors, such as cyclosporine, tacrolimus and sirolimus, or immune modulators, such as azatioprine and mercaptopurine.

Other objectives, features and advantages of the present invention will appear from the following detailed disclosure, from the attached claims, as well as from the figures. It is noted that the invention relates to all possible combinations of features.

Generally, all terms used in the claims are to be interpreted according to their ordinary meaning in the technical field, unless explicitly defined otherwise herein. All references to “a/an/the [step, sample, marker, etc.]” are to be interpreted openly as referring to at least one instance of said step, sample, marker etc., unless explicitly stated otherwise. The steps of any method disclosed herein do not have to be performed in the exact order disclosed, unless explicitly stated.

As used herein, the term “comprising” and variations of that term are not intended to exclude other markers, integers or steps.

### BRIEF DESCRIPTION OF FIGURES

By way of example, embodiments of the present invention will now be described with reference to the accompanying figures, in which:

FIG. 1 shows the proportion of CD19⁺ B-cells which are CD23⁺ (a), the proportion of CD19⁺ B-cells which are CD27⁺ (b), the ratio of CD19⁺ B-cells which are CD23⁺ to CD19⁺ B-cells which are CD27⁺ (c), and the absolute number of CD27⁺ CD19⁺ B cells per mL of blood (d) in peripheral blood samples from patients. The filled squares represent samples from pediatric patients suffering from Crohn's disease (CD, n=8), the filled triangles represent samples from pediatric patients suffering from ulcerative colitis (UC, n=16), and the open circles represent samples from non-IBD control children who had similar symptoms to the children with IBD but no signs of gut inflammation (Ctrl, n=21 in (b) and n=20 in (a), (b) and (c)). The horizontal lines indicate the median values. The nonparametric Kruskal-Wallis test for multiple comparisons was used to statistically analyze differences between the patient groups. A P-value lower than 0.05 was considered significant.

FIG. 2 shows the proportion of CD8⁺ T-cells which are HLA-DR⁺ (a), the proportion of CD4⁺ T-cells which are HLA-DR⁺ (b), the proportion of CD8⁺ T-cells which are CD62L⁺ (c), the ratio of CD8⁺ T-cells which are HLA-DR⁺ to CD8⁺ T-cells which are CD62L⁺ (d), and the absolute number of HLA-DR⁺ CD8⁺ T-cells per mL of blood (e) in peripheral blood samples from patients. The filled squares represent samples from pediatric patients suffering from Crohn's disease (CD, n=8), the filled triangles represent samples from pediatric patients suffering from ulcerative colitis (UC, n=17), and the open circles represent samples from non-IBD control children who had similar symptoms to the children with IBD but no signs of gut inflammation (Ctrl, n=22 in (b), n=19 in (a), (c) and (d)). The nonparametric Kruskal-Wallis test for multiple comparisons was used to statistically analyze differences between the patient groups. A P-value lower than 0.05 was considered significant

FIG. 3 shows the amount of calprotectin (mg/kg faeces) (a), orosomucoid (g/L blood) (b), hemoglobin (g/L blood) (c), the ratio of levels of hemoglobin to levels of orosomucoid (d) and the ratio of levels of hemoglobin to levels of calprotecin (e). The filled squares represent samples from pediatric patients suffering from Crohn's disease (CD, n=8), the filled triangles represent samples from pediatric patients suffering from ulcerative colitis (UC, n=17 (b, c, and d), n=16 (a and e)), and the open circles represent samples from non-IBD control children who had similar symptoms to the children with IBD but no signs of gut inflammation (Ctrl, n=22 in (c), n=21 in (a) and (e), n=20 in (d)). The nonparametric Kruskal-Wallis test for multiple comparisons was used to statistically analyze differences between the patient groups. A P-value lower than 0.05 was considered significant

FIG. 4 shows Module X, an example of how patients with inflammatory bowel disease (IBD, stars) (patients with Crohn's disease or ulcerative colitis) can be separated from controls (Ctrl; being patients with intestinal symptoms not related to IBD, circle), by using A) the levels of faecal calprotectin (threshold; 166.5 mg/kg faeces), B) the levels of orosomucoid (threshold; 0.74 g/L blood), C) the levels of hemoglobin (threshold; 125 g/L blood), D) the ratio hemoglobin/calprotectin (threshold; 0.94), and E) the ratio hemoglobin/orosomucoid (threshold; 168) as biomarkers. Patients with IBD are more likely to have a level above threshold for biomarkers A and B and below threshold for biomarkers C, D, and E, as compared to Ctrls. Filled symbols represent patients that are true positive (TP) or true negative (TN), and unfilled symbols represent patients that are false positive (FP) or false negative (FN). Thus, filled symbols represent correctly diagnosed subjects, whereas unfilled symbols represent subjects that were wrongly diagnosed.

FIG. 5 shows Module Y, an example of how patients with Crohn's disease (CD, square) can be separated from patients with ulcerative colitis (UC, triangle) and controls (Ctrl; being patients with intestinal symptoms not related to IBD, circle), by using A) the proportion of CD27⁺ B-cells (threshold; 16.5%), B) the proportion of CD23⁺ B-cells (threshold; 80%) and C) the ratio CD23⁺/CD27⁺ B-cells (threshold; 5). Patients with CD are more likely to have a value below threshold for biomarker A and above threshold for biomarkers B and C, as compared to UC patients and Ctrls. Filled symbols represent patients that are true positive (TP) or true negative (TN), and unfilled symbols represent patients that are false positive (FP) or false negative (FN). Thus, filled symbols represent correctly diagnosed subjects, whereas unfilled symbols represent subjects that were wrongly diagnosed.

FIG. 6 shows Module Z, an example of how patients with ulcerative colitis (UC, triangle) can be separated from patients with Crohn's disease (CD, square) and controls (Ctrl; being patients with intestinal symptoms not related to IBD, circle), by using A) the proportion of CD8⁺ T cells expressing HLA-DR (threshold; 25.5%), B) the proportion of CD8⁺ T cells expressing CD62L (threshold; 60%), and C) the ratio HLA-DR⁺/CD62L⁺ CD8⁺ T cells (threshold; ratio 0.48) as biomarkers. Patients with UC are more likely to have a ratio above threshold for biomarkers A and C and below threshold for biomarker B, as compared to CD and Ctrl. Filled symbols represent patients that are true positive (TP) or true negative (TN), and unfilled symbols represent patients that are false positive (FP) or false negative (FN). Thus, filled symbols represent correctly diagnosed subjects, whereas unfilled symbols represent subjects that were wrongly diagnosed.

FIG. 7 shows Modules XYZ, an example of how patients with ulcerative colitis (UC, triangle) can be separated from patients with Crohn's disease (CD, square) and controls (Ctrl; being patients with intestinal symptoms not related to IBD, circle), by using a combination of module X (calprotectin, threshold: 166.5 mg/kg faeces), Y (CD27⁺ B cells, threshold: 16.5%) and Z (HLA-DR⁺ CD8⁺ T cells, threshold: 25.5%). Patients with IBD are more likely to have a level above threshold in module X. Patients with CD are more likely to have a proportion below threshold in module Y. Patients with UC are more likely to have a proportion above threshold in module Z. Filled symbols represent patients that are true positive (TP) or true negative (TN), and unfilled symbols represent patients that are false positive (FP) or false negative (FN). Thus, filled symbols represent correctly diagnosed subjects, whereas unfilled symbols represent subjects that were wrongly diagnosed.

FIG. 8 shows Modules ZXY, an example of how patients with ulcerative colitis (UC, triangle) and Crohn's disease (CD, square) can be separated from controls (Ctrl; being patients with intestinal symptoms not related to IBD, circle) by using a combination of module Z (ratio HLA-DR⁺/CD62L⁺ CD8⁺ T cells, threshold: 0.48), X (orosomucoid, threshold: 0.74 g/L) and Y (CD23⁺ B cells, threshold: 80%). Patients with UC are more likely to have a ratio above threshold in module Z. Patients with IBD are more likely to have a level above threshold in module X. Patients with CD are more likely to have a proportion above threshold in module Y. Filled symbols represent patients that are true positive (TP) or true negative (TN), and unfilled symbols represent patients that are false positive (FP) or false negative (FN). Thus, filled symbols represent correctly diagnosed subjects, whereas unfilled symbols represent subjects that were wrongly diagnosed.

FIG. 9 shows Modules YZX, an example of how patients with ulcerative colitis (UC, triangle) and Crohn's disease (CD, square) can be separated from controls (Ctrl; being patients with intestinal symptoms not related to IBD, circle) by using a combination of module Y (ratio CD23⁺/CD27⁺ B cells, threshold: 5), module Z (ratio HLA-DR⁺/CD62L⁺ of CD8⁺ T cells, threshold: 0.48) and module X (ratio hemoglobin/orosmucoid, threshold: 168). Patients with CD are more likely to have a ratio above threshold in module X. Patients with UC are more likely to have a ratio above threshold in module Z. Patients with IBD are more likely to have a ratio below threshold in module X. Filled symbols represent patients that are true positive (TP) or true negative (TN), and unfilled symbols represent patients that are false positive (FP) or false negative (FN). Thus, filled symbols represent correctly diagnosed subjects, whereas unfilled symbols represent subjects that were wrongly diagnosed.

### EXPERIMENTS

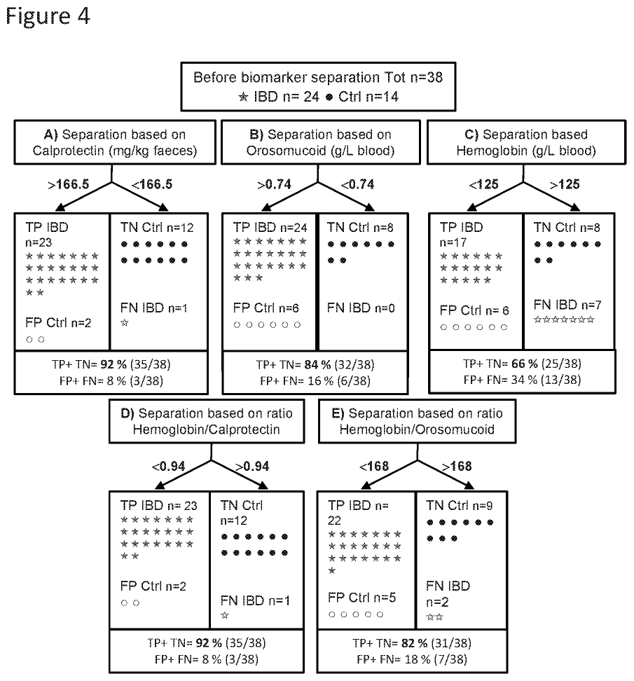

Patients: Children with suspected IBD who were referred to the Pediatric Gastroenterology Unit at the Sahlgrenska University Hospital (Goteborg, Sweden) were eligible for the study. Exclusion criteria were intake of antibiotics, anti-inflammatory drugs or probiotics or any dietary restrictions during the previous 3 months. All the children included in the study underwent a diagnostic work-up, which included esophagogastroduodenoscopy, ileocolonoscopy, and small bowel imaging and were diagnosed according to the Porto criteria (Levine, A., et al., J Pediatr Gastroenterol Nutr, 2014. 58(6): p. 795-806). Blood and fecal samples for the study were obtained before diagnosis and treatment. Twenty-five children, in the age range of 5-16 years and diagnosed with IBD, were included; 17 had ulcerative colitis and 8 had Crohn's disease (Table 1). Children who were found not to have IBD after diagnostic work-up served as symptomatic controls (n=23) (Table 1). These non-IBD controls showed no macroscopic or microscopic signs of inflammation in the gastrointestinal tract and were found to have functional gastrointestinal disorders. Only patients from whom data were available for all biomarkers were included in the modules in FIGS. 4-9. Sixteen of the patients with ulcerative colitis, 8 patients with Crohn's disease, and 14 of the non-IBD controls had complete data sets. Disease distribution and behavior were characterized according to the Paris phenotype classification scheme (Levine, A., et al., Inflamm Bowel Dis, 2011. 17(6): p. 1314-21) (Table 1). The majority of the patients with ulcerative colitis had pancolitis, and colon involvement was also commonly observed in the patients with Crohn's disease (Table 1). None of the patients had perianal disease but one patient presented with orofacial granulomatosis, an inflammatory lesion in the oral mucosa that may be associated with Crohn's disease. All the cases of Crohn's disease showed inflammatory behavior (B1). Six of the children with Crohn's disease displayed granuloma formation at the time of diagnosis. However, within 2 years of the diagnosis being made, granulomas were found in all the patients.

TABLE 1
Clinical characteristics of the patient groups

| | | | Age at | |
|---|---|---|---|---|
| | | Gender | diagnosis | |
| | | distribution | (median, | |
| Diagnosis | N | (boys/girls) | range) | Disease distribution^{a} |
| Ulcerative | 17 | 11/6 | 15 (9-16) | 16 pan-colitis (E4) |
| colitis | | | | 1 proctitis |
| Crohn's | 8 | 4/4 | 11 (6-16) | 5 colon + ileum (L3) |
| disease | | | | 1 colon + ileum + duodenum |
| | | | | (L3, L4a) |
| | | | | 1 colon only (L2) |
| | | | | 1 distal ileum only (L1) + |
| | | | | OFG |
| Controls | 23 | 13/10 | 13 (5-16) | |

^{a}According to the Paris classification (Levine, A., et al., Inflamm Bowel Dis, 2011. 17(6): p. 1314-21).
OFG, orofacial granulomatosis, an oral pathological manifestation associated with Crohn's disease (Kim, S. K. and E. S. Lee, Ann Dermatol, 2010. 22(2): p. 203-5)

Ethics: The study was approved by the Ethics Committee at the University of Gothenburg, Sweden. Written informed consent was obtained from the parents and from older children, whereas younger children provided assent.
Sampling of blood and feces: Before the diagnosis was established and treatment was initiated, a blood sample and a fecal sample were collected from all the patients. The blood samples were analyzed for lymphocyte subsets using flow cytometry and for systemic inflammatory biomarkers, while the samples of feces were analyzed for fecal inflammatory biomarkers.
Inflammatory biomarkers in blood and feces: Analyses of biomarkers of systemic inflammation were carried out at the Clinical Chemistry Laboratory, Sahlgrenska University Hospital. The acute-phase reactant orosomucoid and the negative acute-phase reactant hemoglobin were measured using accredited methodology. As measures of mucosal inflammation, fecal calprotectin, was analyzed at the Clinical Immunology Laboratory, Sahlgrenska University Hospital.
Flow cytometry: Phenotypic analysis of CD4⁺ and CD8⁺ T lymphocytes, as well as of CD19⁺ B cells, was performed by flow cytometry. Whole blood (50 μL/tube for surface staining or 100 μL/tube for surface and intracellular staining) was incubated for 20 min at 4° C. with the anti-human monoclonal antibodies (mAb). Red blood cells were then lysed (FACS Lysing Solution, BD Biosciences), and the remaining cells were washed twice with FACS buffer. To determine the absolute counts of CD4⁺, CD8⁺ and CD19⁺ lymphocytes, the TruCOUNT method was used. Undiluted blood was stained with anti-human CD45 antibody in TruCOUNT tubes (BD Bioscience) and incubated for 15 min at room temperature. The red blood cells were then lysed with FACS Lysing Solution for 15 min at room temperature. All the samples were analyzed within 2 h of staining. Lymphocytes were defined as having low SSC and high expression of CD45, whereas TruCount beads were identified as FL1 and FL2 high. The absolute cell counts for lymphocytes were calculated using the following formula: events of lymphocytes/events of beads multiplied with number of beads per TruCOUNT tube/blood volume. Samples were analyzed in a FacsCalibur (BD Bioscience) equipped with the CellQuestPro software and the data were analyzed using the Flow Jo software (TreeStar, Ashland, Oreg., USA).
Statistical analysis: The non-parametric Kruskal-Wallis test for multiple comparisons was used to analyze differences in expression of surface markers and levels of inflammatory mediators between the different patients groups (GraphPad Prism; GraphPad, San Diego, Calif., USA), as specified in the Figure legends. P<0.05 was considered to be significant (*P<0.05, **P<0.01, ***P<0.001, and ****P<0.0001). In order to determine if the selected markers had a good predictive value, the data was analyzed by performing receiver operator curve (ROC) analysis (GraphPad Prism). In a ROC curve, the true positive rate (sensitivity) is plotted in function of the false positive rate (100-specificity) for different values of a said parameter. The area under the ROC curve (AUC) describes how well a parameter can distinguish between two patient groups, which ranges from 0.5 to 1. A biomarker with no predictive value has an AUC of 0.5, whereas a biomarker with perfect ability to predict outcome would have an AUC of 1. AUC values for the different biomarkers and patient groups are shown in Table 2. The threshold values for the biomarkers were selected based on an optimal value with a sensitivity of at least 80% and/or a specificity of at least 50%, as determined by the ROC analysis. AUC values for the analyzed parameters are shown in Table 2.

TABLE 2
Area under ROC curve analysis

| | Area | | Area | | Area | |
|---|---|---|---|---|---|---|
| | under | | under | | under | |
| | ROC | | ROC | | ROC | |
| | curve | p-value | curve | p-value | curve | p-value |
| Analysis parameter | CD vs UC | | CD vs Ctrl | | CD vs UC and Ctrl | |
| CD23⁺ of CD19⁺ B | 0.67 | Ns | 0.84 | 0.005 | 0.77 | 0.02 |
| cells (%) | | | | | | |
| CD27⁺ of CD19⁺ B | 0.91 | 0.002 | 0.98 | <0.0001 | 0.95 | <0.0001 |
| cells (%) | | | | | | |
| CD23⁺/CD27⁺ | 0.91 | 0.002 | 0.98 | 0.0001 | 0.95 | <0.0001 |
| (ratio) | | | | | | |
| CD27⁺ CD19⁺ B | 0.88 | 0.003 | 0.79 | 0.02 | 0.83 | 0.003 |
| cells (count) | | | | | | |

| | CD vs UC | | UC vs Ctrl | | UC vs CD and Ctrl | |
|---|---|---|---|---|---|---|
| CD62L⁺ of CD8⁺ T | 0.63 | Ns | 0.75 | 0.008 | 0.72 | 0.01 |
| cells (%) | | | | | | |
| HLA-DR⁺ of CD8⁺ T | 0.78 | 0.02 | 0.84 | 0.0006 | 0.82 | 0.0004 |
| cells (%) | | | | | | |
| HLA-DR⁺ of CD4⁺ T | 0.67 | Ns | 0.78 | 0.003 | 0.75 | 0.005 |
| cells (%) | | | | | | |
| HLA-DR⁺/CD62L⁺ | 0.74 | 0.05 | 0.84 | 0.0005 | 0.81 | 0.0005 |
| (ratio) | | | | | | |
| HLA-DR⁺ CD8⁺ T | 0.77 | 0.03 | 0.73 | 0.02 | 0.74 | 0.006 |
| cells (count) | | | | | | |

| | CD vs Ctrl | | UC vs Ctrl | | Ctrl vs CD and UC | |
|---|---|---|---|---|---|---|
| Calprotectin (mg/kg | 0.98 | <0.0001 | 0.91 | <0.0001 | 0.94 | <0.0001 |
| faeces) | | | | | | |
| Orosomucoid(g/L | 0.99 | <0.0001 | 0.89 | <0.0001 | 0.93 | <0.0001 |
| blood) | | | | | | |
| Hemoglobin (g/L | 0.93 | 0.0002 | 0.68 | ns | 0.77 | 0.002 |
| blood) | | | | | | |
| Hemoglobin/ | 0.98 | <0.0001 | 0.91 | <0.0001 | 0.94 | <0.0001 |
| Calprotectin (ratio) | | | | | | |
| Hemoglobin/ | 1 | <0.0001 | 0.91 | <0.0001 | 0.94 | <0.0001 |
| Orosomucoid(ratio) | | | | | | |

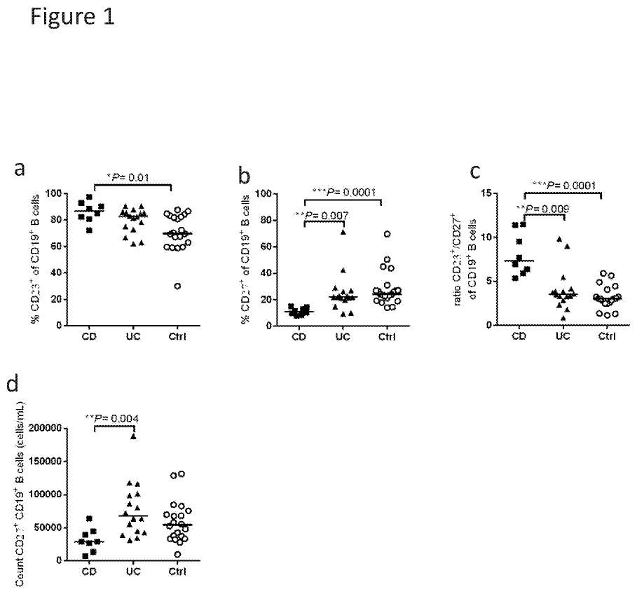

As can be clearly seen in FIG. 1, both the patients suffering from Crohn's disease (CD) and the patients suffering from ulcerative colitis (UC) showed an increase in the proportion of CD19⁺ B-cells which are CD23⁺ compared to the control group (Ctrl) (FIG. 1a), and a decrease in the proportion of CD19⁺ B-cells which are CD27⁺ compared to the control group (Ctrl) (FIG. 1b). FIG. 1c shows that there is a statistically significant difference in the ratio of the proportions of CD23⁺ B-cells and CD27⁺ B-cells between patients suffering from CD or UC compared to the control group. As shown in FIG. 1d, the absolute number of CD27⁺ B cells per ml blood was also significantly lower in patients with CD compared UC. Since the total number of B-cells can vary depending on the health status of the individual (e.g. an individual not suffering from UC or CD has a lower absolute number of B-cells than an individual suffering from UC or CD), the absolute number of CD27⁺ B-cells may display a different pattern compared to when the proportion of B-cells that are CD27⁺ are used as a biomarker.

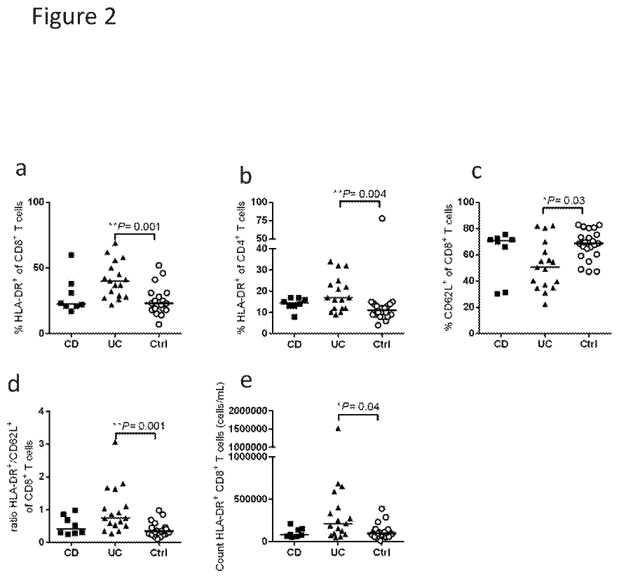

FIG. 2 shows the proportions of CD8⁺ T-cells which are HLA-DR⁺ (FIG. 2a), CD4⁺ T-cells which are HLA-DR⁺ (FIG. 2b) and CD8⁺ T-cells which are CD62L⁺ (FIG. 2c). FIG. 2d shows the ratio of the proportions of HLA-DR⁺ CD8⁺ T-cells and CD62L⁺ CD8⁺ T-cells between patients suffering from CD or UC compared to the control group. As can be seen there is a statistical significant difference between patients suffering from UC and the control group. The absolute number of HLA-DR⁺ CD8⁺ T cells per ml blood was also significantly higher in UC patients compared to controls (FIG. 2e). Thus, the proportions of HLA-DR⁺ CD8⁺ T-cells (FIG. 2a), HLA-DR⁺ CD4⁺ T-cells (FIG. 2b), CD62L⁺ CD8⁺ T-cells (FIG. 2c) or the ratio of the proportions of HLA-DR⁺ CD8⁺ T-cells and CD62L⁺ CD8⁺ T-cells (FIG. 2d) may be used to distinguish patients suffering from UC from patients suffering from CD or patients with IBD-like symptoms. Since the differences in HLA-DR expression also is observed for CD4⁺ T cells (FIG. 2b), which is expressed by the majority of T cells that are not CD8⁺, other markers for T cells, such as the pan T cell marker CD3, can presumably also be used to distinguish patients suffering from UC from patients suffering from CD or patients with IBD-like symptoms.

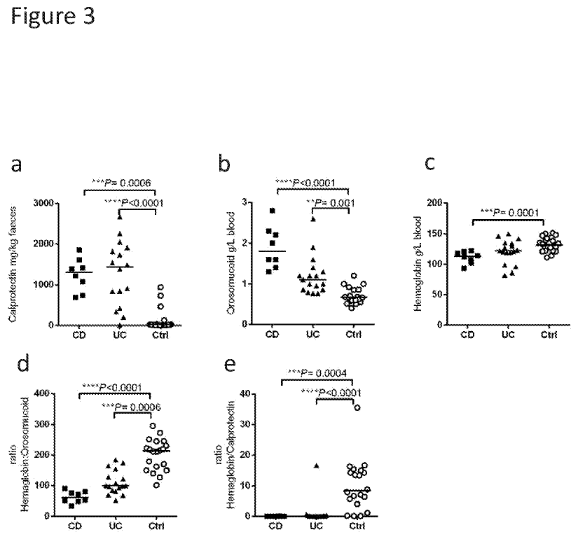

FIG. 3 shows that there is a statistically significant difference between patients suffering from CD or UC and the control group, i.e. patients having IBD-like symptoms, but not suffering from IBD, when comparing the amount of calprotectin in faeces (FIG. 3b) and in the amount of orosomucoid in blood (FIG. 3b). Thus, calprotectin and/or orosomucoid can be used to separate patients suffering from IBD from patients with IBD-like symptoms. As is shown in FIG. 3c, there is a statistically significant difference in the amount of hemoglobin in patients suffering from CD compared to the control group. FIGS. 3d and 3e show that there is a statistically significant difference between patients suffering from CD or UC and the control group, i.e. patients having IBD-like symptoms, but not suffering from IBD, when comparing the ratio of hemoglobin (g/L blood) and orosomucoid (g/L blood) and/or the ratio of hemoglobin (g/L blood) and calprotectin (mg/kg faeces). Thus, the ratio of hemoglobin (g/L blood) and orosomucoid (g/L blood) and/or the ratio of hemoglobin (g/L blood) and calprotectin (mg/kg faeces) can be used to distinguish patients suffering from CD or UC from the control group.

### DETAILED DESCRIPTION OF SPECIFIC EMBODIMENTS

The invention relates to a method for determining if a subject suffers from Crohn's disease or from ulcerative colitis. The subject may be an animal or a human. The subject may be a child, an adolescent or an adult.

In short, it is determined whether the subject suffers from inflammatory bowel disease (IBD) or not and the proportion of B-cells which are CD27⁺ in a sample from the subject is determined. The proportion of B-cells which are CD27⁺ may be used, together with the information whether the subject suffers from inflammatory disease or not, as a means to determine if the subject suffers from Crohn's disease or from ulcerative colitis. Different markers may be used to determine whether the subject suffers from IBD. Such markers include calprotectin, orosomucoid, the ratio of hemoglobin and orosomucoid or the ratio of hemoglobin and calprotectin. If a subject is found to suffer from IBD, a proportion of B-cells which are CD27⁺ which is lower than a threshold value TV_{CD27} of 16.5% indicates that the subject suffers from Crohn's disease. Thus, the conclusion that the subject suffers from Crohn's disease may be drawn. If a subject is found to suffer from IBD, a proportion of B-cells which are CD27⁺ which is higher than a threshold value TV_{CD27} of 16.5% indicates that the subject suffers from ulcerative colitis. Thus, the conclusion that the subject suffers from ulcerative colitis may be drawn.

To further strengthen the indication that a subject suffers from ulcerative colitis, the proportion of CD8⁺ T-cells which are HLA-DR⁺ may be determined in a sample from the subject. If a subject is found to suffer from IBD but not from Crohn's disease, a proportion of CD8⁺ T-cells which are HLA-DR⁺ higher than said threshold value TV_{HLA-DR} of 25.5% indicates that the subject suffers from ulcerative colitis. Thus, the conclusion that the subject suffers from ulcerative colitis may be drawn.

The method may be seen as three different modules, X, Y and Z, which can be combined in different ways.

Module X represents different assays which may be performed in order to distinguish patients suffering from IBD, i.e. Crohn's disease (CD) and/or ulcerative colitis (UC), from patients suffering from similar intestinal symptoms but which are not related to IBD. In FIG. 4, different examples of Module X are shown.

According to FIG. 4A, calprotectin is used to distinguish patients suffering from IBD from patients with similar intestinal symptoms, but not suffering from IBD. An amount higher than 166.5 mg/kg faeces indicates that the patient suffers from IBD. As is shown in FIG. 4A, 92% of the tested patients are correctly diagnosed using this marker.

According to FIG. 4B, orosomucoid is used to distinguish patients suffering from IBD from patients with similar intestinal symptoms, but not suffering from IBD. An amount higher than 0.74 g/L blood indicates that the patient suffers from IBD. As is shown in FIG. 4B, 84% of the tested patients are correctly diagnosed using this marker.

According to FIG. 4C, hemoglobin is used to distinguish patients suffering from IBD from patients with similar intestinal symptoms, but not suffering from IBD. An amount lower than 125 g/L blood indicates that the patient suffers from IBD. As is shown in FIG. 4C, 66% of the tested patients are correctly diagnosed using this marker.

According to FIG. 4D, the ratio of hemoglobin and calprotectin is used to distinguish patients suffering from IBD from patients with similar intestinal symptoms, but not suffering from IBD. A ratio lower than 0.94 indicates that the patient suffers from IBD. As is shown in FIG. 4D, 92% of the tested patients are correctly diagnosed using this marker.

According to FIG. 4E, the ratio of hemoglobin and orosomucoid is used to distinguish patients suffering from IBD from patients with similar intestinal symptoms, but not suffering from IBD. A ratio lower than 168 indicates that the patient suffers from IBD. As is shown in FIG. 4E, 82% of the tested patients are correctly diagnosed using this marker.

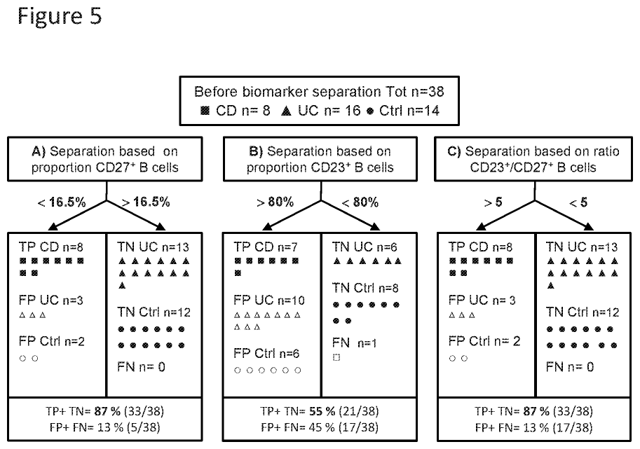

Module Y represents different assays which may be performed in order to distinguish patients suffering from Crohn's disease (CD) from patients suffering from ulcerative colitis (UC) or suffering from similar intestinal symptoms but which are not related to IBD (Ctrl). In FIG. 5, different examples of Module Y are shown.

According to FIG. 5A, the proportion of CD27⁺ B-cells is used to distinguish patients suffering from Crohn's disease (CD) from patients suffering from ulcerative colitis or suffering from similar intestinal symptoms, but not suffering from IBD. A proportion lower than 16.5% indicates that the patient suffers from CD. As is shown in FIG. 5A, 87% of the tested patients are correctly diagnosed using this marker. Alternatively, instead of using the proportion of CD27⁺ B cells, the absolute count of CD27⁺ B cells per ml blood can be used to distinguish patients suffering from Crohn's disease (CD) from patients suffering from ulcerative colitis (UC) or suffering from similar intestinal symptoms but which are not related to IBD (Ctrl). According to FIG. 5B, the proportion of CD23⁺ B-cells is used to distinguish patients suffering from Crohn's disease (CD) from patients suffering from ulcerative colitis or suffering from similar intestinal symptoms, but not suffering from IBD. A proportion higher than 80% indicates that the patient suffers from CD. As is shown in FIG. 5A, 55% of the tested patients are correctly diagnosed using this marker.

According to FIG. 5C, the ratio of CD23⁺ B-cells and CD27⁺ B-cells is used to distinguish patients suffering from Crohn's disease (CD) from patients suffering from ulcerative colitis or suffering from similar intestinal symptoms, but not suffering from IBD. A ratio higher than 5 indicates that the patient suffers from CD. As is shown in FIG. 5C, 87% of the tested patients are correctly diagnosed using this marker.

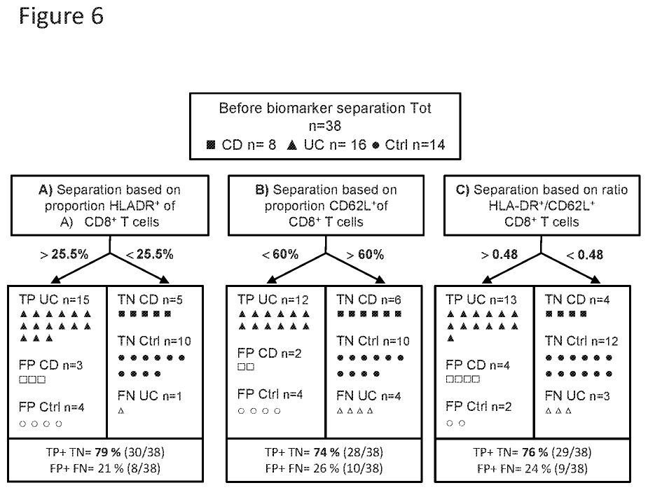

Module Z represents different assays which may be performed in order to distinguish patients suffering from ulcerative colitis (UC) from patients suffering from Crohn's disease (CD) or suffering from similar intestinal symptoms but which are not related to IBD (Ctrl). In FIG. 6, different examples of Module Y are shown.

According to FIG. 6A, the proportion of HLA-DR⁺ CD8⁺ T-cells is used to distinguish patients suffering from ulcerative colitis (UC) from patients suffering from Crohn's disease or suffering from similar intestinal symptoms, but not suffering from IBD. A proportion higher than 25.5% indicates that the patient suffers from UC. As is shown in FIG. 6A, 79% of the tested patients are correctly diagnosed using this marker. Alternatively, the absolute count of HLA-DR⁺ CD8⁺ T cells per ml blood can be used to distinguish patients suffering from ulcerative colitis (UC) from patients suffering from Crohn's disease or suffering from similar intestinal symptoms, but not suffering from IBD.

According to FIG. 6B, the proportion of CD62L⁺ CD8⁺ T-cells is used to distinguish patient suffering from ulcerative colitis (UC) from patients suffering from Crohn's disease or suffering from similar intestinal symptoms, but not suffering from IBD. A proportion lower than 60% indicates that the patient suffers from UC. As is shown in FIG. 6B, 74% of the tested patients are correctly diagnosed using this marker.

According to FIG. 6C, the ratio of HLA-DR⁺ CD8⁺ T-cells and CD62L⁺ CD8⁺ T-cells is used to distinguish patients suffering from ulcerative colitis (UC) from patients suffering from Crohn's disease or suffering from similar intestinal symptoms, but not suffering from IBD. A ratio higher than 0.48% indicates that the patient suffers from UC. As is shown in FIG. 6C, 76% of the tested patients are correctly diagnosed using this marker.

Two or more of the modules X, Y and Z can be combined in order to determine if a subject suffers from Crohn's disease or from ulcerative colitis. Any X module can be combined with any Y module and/or any Z module. Below some specific embodiments are described.

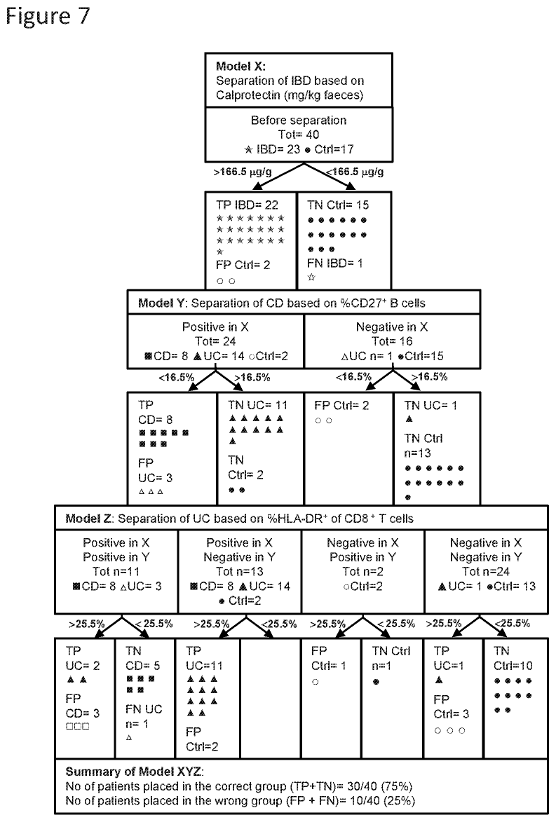

According to the embodiments shown in FIG. 7, if calprotectin is present in an amount higher than 166.5 μg/g faeces, it is likely that the patient suffers from IBD (Module X). A proportion lower than 16.5% of CD27⁺ B-cells in a blood sample from such a patient indicates that the patient suffers from Crohn's disease and a proportion higher than 16.5% of CD27⁺ B-cells in a blood sample from such a patient indicates that the patient suffers from ulcerative colitis. Alternatively, the ratio of CD23⁺ B-cells and CD27⁺ B-cells may be used, wherein a ratio higher than 5 indicates that the patient suffers from Crohn's disease and ratio lower than 5 indicates that the patient suffers from ulcerative colitis.

According to another embodiment (also shown in FIG. 7), the proportion of HLA-DR⁺ CD8⁺ T-cells may be used to further strengthen that a patient suffers from ulcerative colitis. A proportion higher than 25.5% of HLA-DR⁺ CD8⁺ T-cells in a blood sample from such a patient indicates that the patient suffers from ulcerative colitis and a proportion lower than 25.5% of HLA-DR⁺ CD8⁺ T-cells in a blood sample from such a patient indicates that the patient suffers from Crohn's disease. FIG. 7 demonstrates that by using the three biomarkers calprotectin, the proportion of CD27⁺ B-cells, and the proportion HLA-DR⁺ CD8⁺ T-cells, 75% of the tested patients are correctly diagnosed.

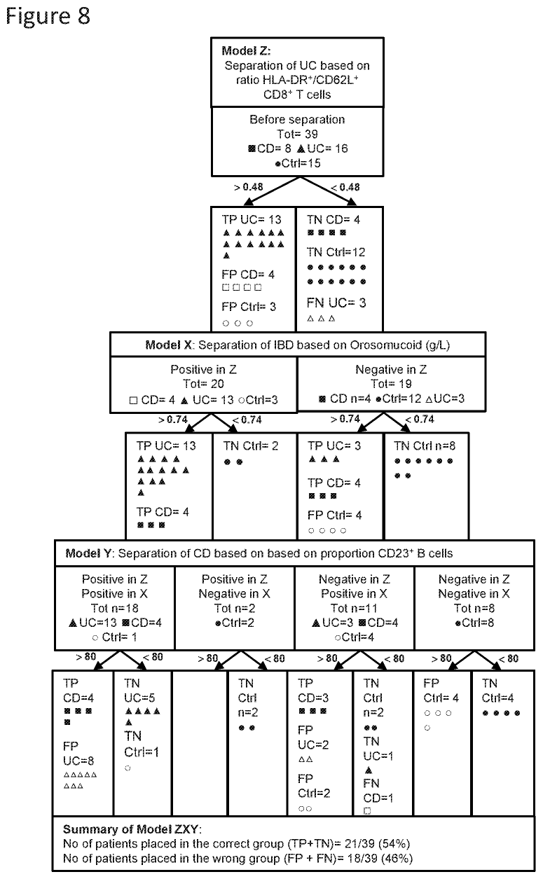

According to the embodiment in FIG. 8, the ratio of HLA-DR⁺ and CD62L⁺ CD8⁺ T cells is used to identify patients suffering from ulcerative colitis (Module Z). A ratio higher than 0.48 indicates that the patient suffers from ulcerative colitis and ratio lower than 0.48 indicates that the patient suffers from Crohn's disease or suffers from similar intestinal symptoms but which are not related to IBD (Ctrl). According to this embodiment, the level of orosomucoid is further used to strengthen the diagnosis. If orosomucoid is present at a level higher than 0.74 g/L blood, it is likely that the patient suffers from IBD (Module X). A proportion lower than 80% of CD23⁺ B-cells in a blood sample from a patient indicates that the patient suffers from ulcerative colitis and a proportion higher than 80% of CD23⁺ B-cells in a blood sample from a patient indicates that the patient suffers from Crohn's disease (Module Y). FIG. 8 demonstrates that by using the three biomarkers ratio of HLA-DR⁺ and CD62L⁺ CD8⁺ T-cells, levels of orosomucoid, and proportion of CD23⁺ B-cells, 54% of the tested patients are correctly diagnosed.

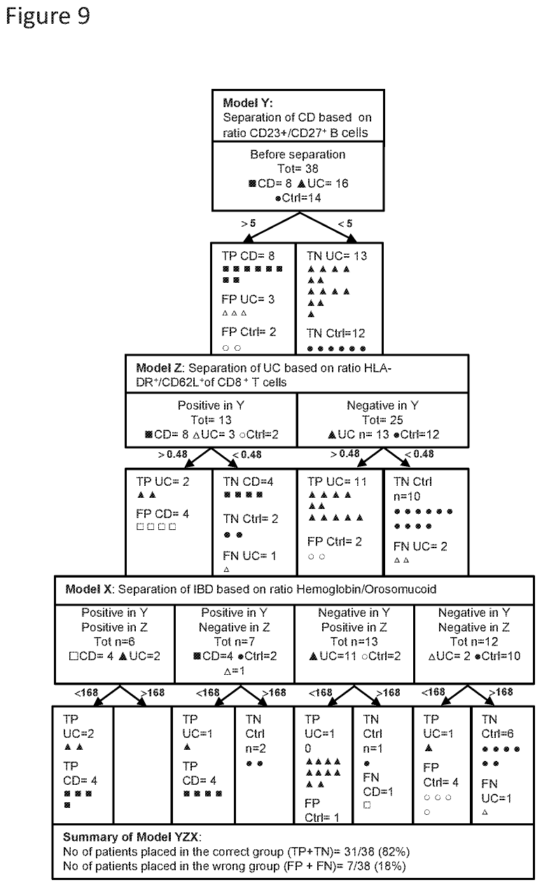

According to the embodiment shown in FIG. 9 the ratio of CD23⁺ B-cells and CD27⁺ B-cells is used to identify patients suffering from Crohn's disease. A ratio higher than 5 indicates that the patient suffers from Crohn's disease and ratio lower than 5 indicates that the patient suffers from ulcerative colitis or suffers from similar intestinal symptoms but which are not related to IBD (Ctrl). According to this invention, the ratio of HLA-DR⁺ CD8⁺ T-cells and CD62L⁺ CD8⁺ T-cells is used to identify patients who suffer from ulcerative colitis. A ratio higher than 0.48 in a blood sample from such a patient indicates that the patient suffers from ulcerative colitis and a proportion lower than 0.48 of HLA-DR⁺ CD8⁺ T-cells in a blood sample from such a patient indicates that the patient suffers from Crohn's disease or suffers from similar intestinal symptoms but which are not related to IBD (Ctrl). According to this embodiment, the ratio of hemoglobin and orosomucoid is further used to strengthen the diagnosis, wherein a ratio below 168 is indicative of the patient suffering from IBD (Crohn's disease or ulcerative colitis) and a ratio above 168 is indicative of the patient suffering from similar intestinal symptoms but which are not related to IBD (Ctrl). FIG. 9 demonstrates that by using the three biomarkers CD23⁺ B-cells/CD27⁺ B-cells HLA-DR⁺/CD62L⁺ CD8⁺ T-cells and hemoglobin/orosmucoid, 82% of the tested patients are correctly diagnosed.

It will be appreciated that the invention has been described with reference to exemplary embodiments, and that the invention can be varied in many different ways within the scope of the claims.

### LIST OF EMBODIMENTS

In the following list of embodiments, a reference made to a particular embodiment number, e.g. embodiment 46, is to be interpreted as a reference to all embodiments encompassing that particular embodiment number, e.g. embodiments 46a, 46b, 46c etc.. Thus, a reference to anyone of embodiments 8 to 12 is to be interpreted as a reference to embodiments 8, 9a, 9b, 10, 11a, 11b and 12.

1. A method for determining if a subject suffers from Crohn's disease or from ulcerative colitis, wherein the method comprises the steps of:
determining if the subject suffers from inflammatory bowel disease;
determining the total number of B-cells which are CD27⁺ in a predetermined volume of a sample S1 from the subject;
comparing said total number with a threshold value TV_{CD27}; and
wherein, if said subject suffers from inflammatory bowel disease, a total number of B-cells which are CD27⁺ in a predetermined volume of a sample lower than said threshold value TV_{CD27} is indicative of that said subject suffers from Crohn's disease; and
wherein, if said subject suffers from inflammatory bowel disease, a total number of B-cells which are CD27⁺ in a predetermined volume of a sample higher than said threshold value TV_{CD27} is indicative of that said subject suffers from ulcerative colitis.

2a. The method according to embodiment 1, wherein the threshold value TV_{CD27} is between 5% and 30%, such as between 10% and 20%, such as between 13% and 18%, such as between 15% and 17%.

2b. The method according to embodiment 1, wherein TV_{CD27} for B-cells is 12%, 14 %, 16.5 %, 18 %, 20 %, or 22 %.

3. The method according to any one of the previous embodiments, wherein the method further comprises the step of
determining the total number of B-cells which are CD23⁺ in a predetermined volume of a sample S2 from the subject;
calculating the ratio of the total numbers of CD23⁺ B-cells and CD27⁺ B-cells;
comparing said ratio with a threshold value TV_{CD23/CD27}; and
wherein, if said subject suffers from inflammatory bowel disease, a ratio higher than said threshold value TV_{CD23/CD27} is indicative of that said subject suffers from Crohn's disease; and
wherein, if said subject suffers from inflammatory bowel disease, a ratio lower than said threshold value TV_{CD23/CD27} is indicative of that said subject suffers from ulcerative colitis.

4a. The method according to embodiment 3, wherein the threshold value TV_{CD23/CD27} is between 0.5 and 8, such as between 2 and 7, such as between 3 and 6, such as between 4 and 5.

4b. The method according to embodiment 3, wherein TV_{CD23/CD27} is 1, 2, 3, 4, 5, 6, or 7.

5. The method according to any one of the previous embodiments, wherein each of the samples S1 and S2 independently is a blood sample or a tissue sample.

6. The method according to any one of the previous embodiments, wherein S1 and S2 are blood.

7. The method according to any one of the previous embodiments, wherein S1 and S2 are the same sample.

8. A method for determining if a subject suffers from Crohn's disease or from ulcerative colitis, wherein the method comprises the steps of:
determining if the subject suffers from inflammatory bowel disease;
determining the proportion of B-cells which are CD27⁺ in a sample S1 from the subject and/or determining the total number of B-cells which are CD27⁺ in a predetermined volume of a sample S1 from the subject;
comparing said proportion with a threshold value TV_{CD27} and/or comparing said total number with a threshold value TV_{CD27}; and
wherein, if said subject suffers from inflammatory bowel disease, a proportion lower than said threshold value TV_{CD27} and/or a total number of B-cells which are CD27⁺ in a predetermined volume of a sample lower than said threshold value TV_{CD27} is indicative of that said subject suffers from Crohn's disease; and
wherein, if said subject suffers from inflammatory bowel disease, a proportion higher than said threshold value TV_{CD27} and/or a total number of B-cells which are CD27⁺ in a predetermined volume of a sample higher than said threshold value TV_{CD27} is indicative of that said subject suffers from ulcerative colitis.

9a. The method according to embodiment 8, wherein the threshold value TV_{CD27} for the proportion of CD27⁺ B cells is between 5% and 30%, such as between 10% and 20%, such as between 13% and 18%, such as between 15% and 17%, and/or the threshold value TV_{CD27} for total number of CD27⁺ B-cells is between 2.5×10⁴ cells/mL blood and 8×10⁴ cells/mL blood, such as between 3×10⁴ cells/mL blood and 7×10⁴ cells/mL blood, such as between 4×10⁴ cells/mL blood and 6.5×10⁴ cells/mL blood, such as between 4.6×10⁴ cells/mL blood and 5.8×10⁴ cells/mL blood.

9b. The method according to embodiment 8, wherein the threshold value TV_{CD27} for the proportion of CD27⁺ B-cells is 12%, 14%, 16.5%, 18%, 20% or 22%, and/or the threshold value TV_{CD27} for the total numbers of CD27⁺ B-cells is 3×10⁴ cells/mL blood, 4×10⁴ cells/mL blood, 4.5×10⁴ cells/mL blood, 5×10⁴ cells/mL blood, 5.5×10⁴ cells/mL blood, or 6×10⁴ cells/mL blood.

10. The method according to embodiment 8, 9a or 9b, wherein the method further comprises the step of
determining the proportion of B-cells which are CD23⁺ in a sample S2 from the subject and/or determining the total number of B-cells which are CD23⁺ in a predetermined volume of a sample S2 from the subject;
calculating the ratio of the proportions of CD23⁺ B-cells and CD27⁺ B-cells and/or calculating the ratio of the total numbers of CD23⁺ B-cells and CD27⁺ B-cells;
comparing at least one of said ratios with a threshold value TV_{CD23/CD27}; and
wherein, if said subject suffers from inflammatory bowel disease, a ratio higher than said threshold value TV_{CD23/CD27} is indicative of that said subject suffers from Crohn's disease; and
wherein, if said subject suffers from inflammatory bowel disease, a ratio lower than said threshold value TV_{CD23/CD27} is indicative of that said subject suffers from ulcerative colitis.

11a. The method according to embodiment 10, wherein the threshold value TV_{CD23/CD27} is between 0.5 and 8, such as between 2 and 7, such as between 3 and 6, such as between 4 and 5.

11b. The method according to embodiment 10, wherein the threshold value TV_{CD23/CD27} is 1, 2, 3, 4, 5, 6, or 7.

12. The method according to any one of embodiments 8, 9a, 9b, 10, 11a and 11b, wherein each of the samples S1 and S2 independently is a blood sample or a tissue sample.

13. The method according to any one of embodiments 8 to 12, wherein S1 and S2 are blood.

14. The method according to any one of embodiments 8 to 13, wherein S1 and S2 are the same sample.

15. A method for determining if a subject suffers from Crohn's disease or from ulcerative colitis, wherein the method comprises the steps of:
determining that the subject suffers from inflammatory bowel disease;
determining the proportion of B-cells which are CD23⁺ in a sample S2 from the subject;
determining the proportion of B-cells which are CD27⁺ in a sample S1 from the subject;
calculating the ratio of the proportions of CD23⁺ B-cells and CD27⁺ B-cells;
comparing said ratio with a threshold value TV_{CD23/CD27}; and
wherein, if said subject suffers from inflammatory bowel disease, a ratio higher than said threshold value TV_{CD23/CD27} is indicative of that said subject suffers from Crohn's disease; and
wherein, if said subject suffers from inflammatory bowel disease, a ratio lower than said threshold value TV_{CD23/CD27} is indicative of that said subject suffers from ulcerative colitis.

16a. The method according to embodiment 15, wherein the threshold value TV_{CD23/CD27} based on proportion of CD23⁺ and CD27⁺ B cells is between 0.5 and 8, such as between 2 and 7, such as between 3 and 6, such as between 4 and 5.

16b. The method according to embodiment 15, wherein TV_{CD23/CD27} is 1, 2, 3, 4, 5, 6, or 7.

17. The method according to embodiment 15, 16a or 16b, wherein the samples S2 and S1 independent of each other are chosen from a blood sample or a tissue sample.

18. The method according to any one of embodiments 15 to 17, wherein S1 and S2 are blood.

19. The method according to any one of embodiments 15 to 18, wherein S1 and S2 are the same sample.

20. A method for determining if a subject suffers from Crohn's disease or from ulcerative colitis, wherein the method comprises the steps of:
determining that the subject suffers from inflammatory bowel disease;
determining the total number of B-cells which are CD23⁺ in a predetermined volume of a sample S2 from the subject;
determining the total number of B-cells which are CD27⁺ in a predetermined volume of a sample S1 from the subject;
calculating the ratio of the total number of CD23⁺ B-cells and CD27⁺ B cells;
comparing said ratio with a threshold value TV_{CD23/CD27}; and
wherein, if said subject suffers from inflammatory bowel disease, a ratio higher than said threshold value TV_{CD23/CD27} is indicative of that said subject suffers from Crohn's disease; and
wherein, if said subject suffers from inflammatory bowel disease, a ratio lower than said threshold value TV_{CD23/CD27} is indicative of that said subject suffers from ulcerative colitis.

21a. The method according to embodiment 20, wherein the threshold value TV_{CD23/CD27} based on the total number of B cells that are CD23⁺ and CD27⁺ B cells is between 0.5 and 8, such as between 2 and 7, such as between 3 and 6, such as between 4 and 5.

21b. The method according to embodiment 20, wherein TV_{CD23/CD27} is 1, 2, 3, 4, 5, 6, or 7.

22. The method according to embodiment 20, 21a or 21b, wherein the samples S2 and S1 independent of each other are chosen from a blood sample or a tissue sample.

23. The method according to any one of embodiments 20 to 22, wherein S1 and S2 are blood.

24. The method according to any one of embodiments 20 to 23, wherein S1 and S2 are the same sample.

25. A method for determining if a subject suffers from Crohn's disease or from ulcerative colitis, wherein the method comprises the steps of:
determining that the subject suffers from inflammatory bowel disease;
determining the proportion of B-cells which are CD23⁺ in a sample S2 from the subject and/or determining the total number of B-cells which are CD23⁺ in a predetermined volume of a sample S2 from the subject;
determining the proportion of B-cells which are CD27⁺ in a sample S1 from the subject and/or determining the total number of B-cells which are CD27⁺ in a predetermined volume of a sample S1 from the subject;
calculating the ratio of the proportions of CD23⁺ B-cells and CD27⁺ B-cells and or calculating the ratio of the total number of CD23⁺ B-cells and CD27⁺ B-cells;
comparing at least one of said ratios with a threshold value TV_{CD23/CD27}; and
wherein, if said subject suffers from inflammatory bowel disease, a ratio higher than said threshold value TV_{CD23/CD27} is indicative of that said subject suffers from Crohn's disease; and
wherein, if said subject suffers from inflammatory bowel disease, a ratio lower than said threshold value TV_{CD23/CD27} is indicative of that said subject suffers from ulcerative colitis.

26a. The method according to embodiment 25, wherein the threshold value TV_{CD23/CD27} based on proportion and/or total number of CD23⁺ and CD27⁺ B cells is between 0.5 and 8, such as between 2 and 7, such as between 3 and 6, such as between 4 and 5.

26b. The method according to embodiment 25, wherein TV_{CD23/CD27} is 1, 2, 3, 4, 5, 6, or 7.

27. The method according to embodiment 25, 26a or 26b, wherein the samples S2 and S1 independent of each other are chosen from a blood sample or a tissue sample.

28. The method according to any one of embodiments 25 to 27, wherein S1 and S2 are blood.

29. The method according to any one of embodiments 25 to 28, wherein S1 and S2 are the same sample.

30. Method according to any one of the previous embodiments, wherein the step of determining that the subject suffers from inflammatory bowel disease comprises:
determining the amount of hemoglobin in a sample S3 and the amount of orosomucoid in a sample S4 from the subject;
calculating the ratio of the amount of hemoglobin in the sample S3 and the amount of orosomucoid in the sample S4;
comparing said ratio with a threshold value TV_{H/O};
wherein, a ratio lower than said threshold value TV_{H/O} is indicative of that said subject suffers from inflammatory bowel disease.

31a. The method according to embodiment 30, wherein the threshold value TV_{H/O} is between 100 and 200, such as between 125 and 190, such as between 140 and 180, such as between 150 and 170.

31b. The method according to embodiment 30, wherein TV_{H/O} is 120, 130, 140, 150, 160, 168, 170, or 180.

32. The method according to embodiment 30, 31a or 31b, wherein S3 and S4 are blood.

33. The method according to any one of embodiments 30 to 32, wherein S3 and S4 are the same sample.

34. The method according to any one of embodiments 30 to 33, wherein at least two of the samples S1, S2, S3 and S4 are the same sample.

35. Method according to any one of embodiments 1 to 34, wherein the step of determining that the subject suffers from inflammatory bowel disease comprises:
determining the amount of hemoglobin in a sample S3 and calprotectin in a sample S5 from the subject;
calculating the ratio of the amounts of hemoglobin in the sample S3 and calprotectin in the sample S5;
comparing said ratio with a threshold value TV_{H/C};
wherein, a ratio lower than said threshold value TV_{H/C} is indicative of that said subject suffers from inflammatory bowel disease.

36a. The method according to embodiment 35, wherein the threshold value TV_{H/C} is between 0.1 and 5, such as between 0.2 and 4, such as between 0.4 and 3, such as between 0.6 and 2, such as between 0.8 and 1.

36b. The method according to embodiment 35, wherein TV_{H/C} is 0.4, 0.6, 0.8, 0.94, 1, 2, 3, or 4.

37. The method according to embodiment 35, 36a or 36b, wherein the sample S3 is blood and wherein the sample S5 is faeces.

38. The method according to any one of embodiments 35 to 37, wherein at least two of the samples S1, S2 and S3 are the same sample.

39. Method according to any one of embodiments 1 to 34, wherein the step of determining that the subject suffers from inflammatory bowel disease comprises the steps of:
determining the amount of orosomucoid in a sample S4 from the subject;
comparing said amount of orosomucoid with a threshold value for orosomucoid TV_{O};
wherein, an amount of orosomucoid higher than said threshold value TV_{O} is indicative of that said subject suffers from inflammatory bowel disease.

40a. The method according to embodiment 39, wherein the threshold value TV_{O} is between 0.5 and 1 g/L blood, such as between 0.6 and 0.9 g/L blood, such as between 0.65 and 0.85 g/L blood, such as between 0.7 and 0.8 g/L blood.

40b. The method according to embodiment 39, wherein TV_{O} is 0.5 g/L blood, 0.6 g/L blood, 0.7 g/L blood, 0.74 g/L blood, 0.8 g/L blood, 0.9 g/L blood, or 1 g/L blood.

41. The method according to embodiment 39, 40a or 40b, wherein the sample S4 is blood.

42. The method according to any one of embodiments 39 to 41, wherein at least two of the samples S1, S2 and S4 are the same sample.

43. Method according to any one of embodiments 1 to 34, wherein the step of determining that the subject suffers from inflammatory bowel disease comprises the steps of:
determining the amount of calprotectin in a sample S5 from the subject;
comparing said amount of calprotectin with a threshold value TV_{C} for calprotectin;
wherein, an amount of calprotectin higher than said threshold value TV_{C} is indicative of that said subject suffers from inflammatory bowel disease.

44a. The method according to embodiment 43, wherein the threshold value TV_{C} is between 50 and 300 mg/kg faeces, such as between 75 and 250 mg/kg faeces, such as between 100 and 200 mg/kg faeces, such as between 130 and 175 mg/kg faeces, such as between 140 and 170 mg/kg faeces.

44b. The method according to embodiment 43, wherein TV_{C} is 75 mg/kg faeces, 100 mg/kg faeces, 120 mg/kg faeces, 140 mg/kg faeces, 150 mg/kg faeces, 160 mg/kg faeces, 166.5 mg/kg faeces, 170 mg/kg faeces, 180 mg/kg faeces, 200 mg/kg faeces, or 220 mg/kg faeces.

45. The method according to embodiment 43, 44a or 44b, wherein the sample S5 is faeces.

46a. Method according to any one of the previous embodiments, wherein the method further comprises the steps of:
determining the proportion of CD8⁺ T-cells which are HLA-DR⁺ in a sample S6 from the subject;
comparing said proportion with a threshold value TV_{HLA-DR(CD8+)P};
wherein, a proportion of CD8⁺ T-cells which are HLA-DR⁺ higher than said threshold value TV_{HLA-DR(CD8+)P} is indicative of that said subject suffers from ulcerative colitis.

46b. Method according to any one of the previous embodiments, wherein the method further comprises the steps of:
determining the proportion of CD4⁺ T-cells which are HLA-DR⁺ in a sample S6 from the subject;
comparing said proportion with a threshold value TV_{HLA-DR(CD4+)P};
wherein, a proportion of CD4⁺ T-cells which are HLA-DR⁺ higher than said threshold value TV_{HLA-DR(CD4+)P} is indicative of that said subject suffers from ulcerative colitis.

46c. Method according to any one of the previous embodiments, wherein the method further comprises the steps of:
determining the proportion of CD3⁺ T-cells which are HLA-DR⁺ in a sample S6 from the subject;
comparing said proportion with a threshold value TV_{HLA-DR(CD3+)P};
wherein, a proportion of CD3⁺ T-cells which are HLA-DR⁺ higher than said threshold value TV_{HLA-DR(CD3+)P} is indicative of that said subject suffers from ulcerative colitis.

47a. The method according to embodiment 46a, wherein the threshold value TV_{HLA-DR(CD8+)P} is between 10 and 40%, such as between 15 and 30%, such as between 20 and 28%, such as between 22 and 26%.

47b. The method according to embodiment 46a, wherein the threshold value TV_{HLA-DR(CD8+)P} is 12%, 15%, 17%, 20%, 22%, 25.5%, 27%, 30% or 35%.

47c. The method according to embodiment 46b, wherein the threshold value TV_{HLA-DR(CD4+)P} is between 10 and 40%, such as between 15 and 30%, such as between 20 and 28%, such as between 22 and 26%.

47d. The method according to embodiment 46b, wherein the threshold value TV_{HLA-DR(CD4+)P} is 12%, 15%, 17%, 20%, 22%, 25.5%, 27%, 30% or 35%. 47e. The method according to embodiment 46c, wherein the threshold value TV_{HLA-DR(CD3+)P} is between 10 and 40%, such as between 15 and 30%, such as between 20 and 28%, such as between 22 and 26%.

47f. The method according to embodiment 46b, wherein the threshold value TV_{HLA-DR(CD3+)P} is 12%, 15%, 17%, 20%, 22%, 25.5%, 27%, 30% or 35%.

48. The method according to embodiment 46a, 46b, 46c, 47a, 47b, 47c, 47d, 47e, or 47f wherein the sample S6 is blood.

49. The method according to any one of embodiments 46a, 46b, 46c, 47a, 47b, 47c, 47d, 47e, 47f or 48, wherein at least two of the samples S1, S2, S3, S4 and S6 are the same sample.

50a. Method according to any one of embodiments 1 to 45, wherein the method further comprises the steps of:
determining the total number of CD8⁺ T-cells which are HLA-DR⁺ in a predetermined volume of a sample S6 from the subject;
comparing said total number with a threshold value TV_{HLA-DR(CD8+)N};
wherein, a total number of CD8⁺ T-cells which are HLA-DR⁺ higher than said threshold value TV_{HLA-DR(CD8+)N} is indicative of that said subject suffers from ulcerative colitis.

50b. Method according to any one of embodiments 1 to 45, wherein the method further comprises the steps of:
determining the total number of CD4⁺ T-cells which are HLA-DR⁺ in a predetermined volume of a sample S6 from the subject;
comparing said total number with a threshold value TV_{HLA-DR(CD4+)N};
wherein, a total number of CD4⁺ T-cells which are HLA-DR⁺ higher than said threshold value TV_{HLA-DR(CD4+)N} is indicative of that said subject suffers from ulcerative colitis.

50c. Method according to any one of embodiments 1 to 45, wherein the method further comprises the steps of:
determining the total number of CD3⁺ T-cells which are HLA-DR⁺ in a predetermined volume of a sample S6 from the subject;
comparing said total number with a threshold value TV_{HLA-DR(CD3+)N};
wherein, a total number of CD3⁺ T-cells which are HLA-DR⁺ higher than said threshold value TV_{HLA-DR(CD3+)N} is indicative of that said subject suffers from ulcerative colitis.

51a. The method according to embodiment 50a, wherein the threshold value TV_{HLA-DR(CD8+)N} is between 0.5×10⁵ and 3×10⁵ cells/mL blood, such as between 1×10⁵ and 2×10⁵ cells/mL blood, such as between 1.5×10⁵ and 1.8×10⁵ cells/mL blood.

51b. The method according to embodiment 50a, wherein TV_{HLA-DR(CD8+)N} is 0.5×10⁵ cells/mL blood, 1×10⁵ cells/mL blood, 1.5×10⁵ cells/mL blood, 2×10⁵ cells/mL blood, or 2.5×10⁵ cells/mL blood.

51c. The method according to embodiment 50b, wherein the threshold value TV_{HLA-DR(CD4+)N} is between 0.5×10⁵ and 3×10⁵ cells/mL blood, such as between 1×10⁵ and 2×10⁵ cells/mL blood, such as between 1.5×10⁵ and 1.8×10⁵ cells/mL blood.

51d. The method according to embodiment 50b, wherein TV_{HLA-DR(CD4+)N} is 0.5×10⁵ cells/mL blood, 1×10⁵ cells/mL blood, 1.5×10⁵ cells/mL blood, 2×10⁵ cells/mL blood, or 2.5×10⁵ cells/mL blood.

51e. The method according to embodiment 50c, wherein the threshold value TV_{HLA-DR(CD3+)N} is between 0.5×10⁵ and 3×10⁵ cells/mL blood, such as between 1×10⁵ and 2×10⁵ cells/mL blood, such as between 1.5×10⁵ and 1.8×10⁵ cells/mL blood.

51f. The method according to embodiment 50c, wherein TV_{HLA-DR(CD3+)N} is 0.5×10⁵ cells/mL blood, 1×10⁵ cells/mL blood, 1.5×10⁵ cells/mL blood, 2×10⁵ cells/mL blood, or 2.5×10⁵ cells/mL blood.

52. The method according to embodiment 50a, 50b, 50c, 51a, 51b, 51c, 51d, 51e or 51f, wherein the sample S6 is blood.

53. The method according to any one of embodiments 50a, 50b, 50c, 51a, 51b, 51c, 51d, 51e, 51f, or 52, wherein at least two of the samples S1, S2, S3, S4, and S6 are the same sample.

54a. Method according to any one of embodiments 1 to 45, wherein the method further comprises the steps of:
determining the proportion of CD8⁺T-cells which are HLA-DR⁺ in a sample S6 from the subject and/or determining the total number of CD8⁺-T-cells which are HLA-DR⁺ in predetermined volume of a sample S6 from the subject;
comparing said proportion with a threshold value TV_{HLA-DR(CD8+)P} and/or comparing said total number with a threshold value TV_{HLA-DR(CD8+)N};
wherein, a proportion of CD8⁺T-cells which are HLA-DR⁺ higher than said threshold value TV_{HLA-DR(CD8+)P} and/or a total number of CD8⁺ T-cells which are HLA-DR⁺ lower than said threshold value TV_{HLA-DR(CD8+)N} is indicative of that said subject suffers from ulcerative colitis.

54b. Method according to any one of embodiments 1 to 45, wherein the method further comprises the steps of:
determining the proportion of CD4⁺ T-cells which are HLA-DR⁺ in a sample S6 from the subject and/or determining the total number of CD4⁺ T-cells which are HLA-DR⁺ in predetermined volume of a sample S6 from the subject;
comparing said proportion with a threshold value TV_{HLA-DR(CD4+)P} and/or
comparing said total number with a threshold value TV_{HLA-DR(CD4+)N};
wherein, a proportion of CD4⁺ T-cells which are HLA-DR⁺ higher than said threshold value TV_{HLA-DR(CD4+)P} and/or a total number of CD4⁺ T-cells which are HLA-DR⁺ higher than said threshold value TV_{HLA-DR(CD4+)N} is indicative of that said subject suffers from ulcerative colitis.

54c. Method according to any one of embodiments 1 to 45, wherein the method further comprises the steps of:
determining proportion of CD3⁺ T-cells which are HLA-DR⁺ in a sample S6 from the subject and/or determining the total number of total CD3⁺ T cells which are HLA-DR⁺ in predetermined volume of a sample S6 from the subject;
comparing said proportion with a threshold value TV_{HLA-DR(CD3+)P} and/or comparing said total number with a threshold value TV_{HLA-DR(CD3+)N};
wherein, a proportion of CD3+T-cells which are HLA-DR⁺ higher than said threshold value TV_{HLA-DR(CD3+)P} and/or a total number of CD3⁺ T-cells which are HLA-DR⁺ higher than said threshold value TV_{HLA-DR(CD3+)N} is indicative of that said subject suffers from ulcerative colitis.

55a. The method according to embodiment 54a, wherein the threshold value TV_{HLA-DR(CD8+)P} is between 10% and 40%, such as between 15% and 30%, such as between 20% and 28%, such as between 22% and 26%, and/or the threshold value TV_{HLA-DR(CD8+)N} is between 0.5×10⁵ and 3×10⁵ cells/mL blood, such as between 1×10⁵ and 2×10⁵ cells/mL blood, such as between 1.5×10⁵ and 1.8×10⁵ cells/mL blood.

55b. The method according to embodiment 54a, wherein TV_{HLA-DR(CD8+)P} is 12%, 15%, 17%, 20%, 22%, 25.5%, 27%, 30% or 35% and/or TV_{HLA-DR(CD8+)N} is 0.5×10⁵ cells/mL blood, 1×10⁵ cells/mL blood, 1.5×10⁵ cells/mL blood, 2×10⁵ cells/mL blood, or 2.5×10⁵ cells/mL blood.

55c. The method according to embodiment 54b, wherein the threshold value TV_{HLA-DR(CD4+)P} is between 10% and 40%, such as between 15% and 30%, such as between 20% and 28%, such as between 22% and 26%, and/or the threshold value TV_{HLA-DR(CD4+)N} is between 0.5×10⁵ and 3×10⁵ cells/mL blood, such as between 1×10⁵ and 2×10⁵ cells/mL blood, such as between 1.5×10⁵ and 1.8×10⁵ cells/mL blood.

55d. The method according to embodiment 54b, wherein TV_{HLA-DR(CD4+)P} is 12%, 15%, 17%, 20%, 22%, 25.5%, 27%, 30% or 35% and/or TV_{HLA-DR(CD4+)N} is 0.5×10⁵ cells/mL blood, 1×10⁵ cells/mL blood, 1.5×10⁵ cells/mL blood, 2×10⁵ cells/mL blood, or 2.5×10⁵ cells/mL blood.

55e. The method according to embodiment 54c, wherein the threshold value TV_{HLA-DR(CD3+)P} is between 10% and 40%, such as between 15% and 30%, such as between 20% and 28%, such as between 22% and 26%, and/or the threshold value TV_{HLA-DR(CD3+)N} is between 0.5×10⁵ and 3×10⁵ cells/mL blood, such as between 1×10⁵ and 2×10⁵ cells/mL blood, such as between 1.5×10⁵ and 1.8×10⁵ cells/mL blood.

55f. The method according to embodiment 54c, wherein TV_{HLA-DR(CD3+)P} is 12%, 15%, 17%, 20%, 22%, 25.5%, 27%, 30% or 35% and/or TV_{HLA-DR(CD3+)N} is 0.5×10⁵ cells/mL blood, 1×10⁵ cells/mL blood, 1.5×10⁵ cells/mL blood, 2×10⁵ cells/mL blood, or 2.5×10⁵ cells/mL blood.

56. The method according to embodiment 54a, 54b, 54c, 55a, 55b, 55c, 55d, 55e, or 55f wherein the sample S6 is blood.

57. The method according to any one of embodiments 54a, 54b, 54c, 55a, 55b, 55c, 55d, 55e, 55f or 56, wherein at least two of the samples S1, S2, S3, S4, and S6 are the same sample.

58a. Method according to any one of the embodiments 1 to 45, wherein the method further comprises the steps of:
determining the proportion of CD8⁺ T-cells which are HLA-DR⁺ in a sample S6 from the subject;
determining the proportion of CD8⁺ T-cells which are CD62L⁺ in a sample S7 from the subject;
calculating the ratio of the proportions of HLA-DR⁺ CD8⁺ T-cells and CD62L⁺ CD8⁺ T-cells;
comparing said ratio with a threshold value TV_{HLA-DR/CD62L(CD8+)P}; and
wherein, if said subject suffers from inflammatory bowel disease, a ratio higher than said threshold value TV_{HLA-DR/CD62L(CD8+)P} is indicative of that said subject suffers from ulcerative colitis.

58b. Method according to any one of the embodiments 1 to 45, wherein the method further comprises the steps of:
determining the proportion of CD4⁺ T-cells which are HLA-DR⁺ in a sample S6 from the subject;
determining the proportion of CD4⁺ T-cells which are CD62L⁺ in a sample S7 from the subject;
calculating the ratio of the proportions of HLA-DR⁺ CD4⁺ T-cells and CD62L⁺ CD4⁺ T-cells;
comparing said ratio with a threshold value TV_{HLA-DR/CD62L(CD4+)P}; and
wherein, if said subject suffers from inflammatory bowel disease, a ratio higher than said threshold value TV_{HLA-DR/CD62L(CD4+)P} is indicative of that said subject suffers from ulcerative colitis.

58c. Method according to any one of the embodiments 1 to 45, wherein the method further comprises the steps of:
determining the proportion of CD3⁺ T-cells which are HLA-DR⁺ in a sample S6 from the subject;
determining the proportion of CD3⁺ T-cells which are CD62L⁺ in a sample S7 from the subject;
calculating the ratio of the proportions of HLA-DR⁺ CD3⁺ T-cells and CD62L⁺ CD3⁺ T-cells;
comparing said ratio with a threshold value TV_{HLA-DR/CD62L(CD3+)P}; and
wherein, if said subject suffers from inflammatory bowel disease, a ratio higher than said threshold value TV_{HLA-DR/CD62L(CD3+)P} is indicative of that said subject suffers from ulcerative colitis.

59a. The method according to embodiment 58a, wherein the threshold value TV_{HLA-DR/CD62L(CD8+)P} is between 0.2 and 0.8, such as between 0.3 and 0.7, such as between 0.4 and 0.6, such as between 0.45 and 0.5.

59b. The method according to embodiment 58a, wherein the threshold value TV_{HLA-DR/CD62L(CD8+)P} is 0.3, 0.35, 0.4, 0.45, 0.48, 0.5, 0.6 or 0.7.

59c. The method according to embodiment 58b, wherein the threshold value TV_{HLA-DR/CD62L(CD4+)P}is between 0.2 and 0.8, such as between 0.3 and 0.7, such as between 0.4 and 0.6, such as between 0.45 and 0.5.

59d. The method according to embodiment 58b, wherein the threshold value TV_{HLA-DR/CD62L(CD4+)P}is 0.3, 0.35, 0.4, 0.45, 0.48, 0.5, 0.6 or 0.7.

59e. The method according to embodiment 58c, wherein the threshold value TV_{HLA-DR/CD62L(CD3+)P} is between 0.2 and 0.8, such as between 0.3 and 0.7, such as between 0.4 and 0.6, such as between 0.45 and 0.5.

59f. The method according to embodiment 58c, wherein the threshold value TV_{HLA-DR/CD62L(CD3+)P} is 0.3, 0.35, 0.4, 0.45, 0.48, 0.5, 0.6 or 0.7.

60. The method according to embodiment 58a, 58b, 58c, 59a, 59b, 59c, 59d, 59e or 59f wherein the samples S6 and S7 are blood.

61. The method according to any one of embodiments 58a, 58b, 58c, 59a, 59b, 59c, 59d, 59e, 59f or 60, wherein at least two of the samples S1, S2, S3, S4, S6 and S7 are the same sample.

62a. The method according to any one of the embodiments 1 to 45, wherein the method further comprises the steps of:
determining the total number of CD8⁺ T-cells which are HLA-DR⁺ in a predetermined volume of a sample S6 from the subject;
determining the total number of CD8⁺ T-cells which are CD62L⁺ in a predetermined volume of a sample S7 from the subject;
calculating the ratio of the total numbers of HLA-DR⁺ CD8⁺ T-cells and CD62L⁺ CD8⁺ T-cells;
comparing said ratio with a threshold value TV_{HLA-DR/CD62L(CD8+)N}; and
wherein, if said subject suffers from inflammatory bowel disease, a ratio higher than said threshold value TV_{HLA-DR/CD62L(CD8+)N} is indicative of that said subject suffers from ulcerative colitis.

62b. The method according to any one of the embodiments 1 to 45, wherein the method further comprises the steps of:
determining the total number of CD4⁺ T-cells which are HLA-DR⁺ in a predetermined volume of a sample S6 from the subject;
determining the total number of CD4⁺ T-cells which are CD62L⁺ in a predetermined volume of a sample S7 from the subject;
calculating the ratio of the total numbers of HLA-DR⁺ CD4⁺ T-cells and CD62L⁺ CD4⁺ T-cells;
comparing said ratio with a threshold value TV_{HLA-DR/CD62L(CD4+)N}; and
wherein, if said subject suffers from inflammatory bowel disease, a ratio higher than said threshold value TV_{HLA-DR/CD62L(CD4+)N} is indicative of that said subject suffers from ulcerative colitis.

62c. The method according to any one of the embodiments 1 to 45, wherein the method further comprises the steps of:
determining the total number of CD3⁺ T-cells which are HLA-DR⁺ in a predetermined volume of a sample S6 from the subject;
determining the total number of CD3⁺ T-cells which are CD62L⁺ in a predetermined volume of a sample S7 from the subject;
calculating the ratio of the total numbers of HLA-DR⁺ CD3⁺ T-cells and CD62L⁺ CD3⁺ T-cells;
comparing said ratio with a threshold value TV_{HLA-DR/CD62L(CD3+)N}; and
wherein, if said subject suffers from inflammatory bowel disease, a ratio higher than said threshold value TV_{HLA-DR/CD62L(CD3+)N} is indicative of that said subject suffers from ulcerative colitis.

63a. The method according to embodiment 62a, wherein the threshold value TV_{HLA-DR/CD62L(CD8+)N} is between 0.2 and 0.8, such as between 0.3 and 0.7, such as between 0.4 and 0.6, such as between 0.45 and 0.5.

63b. The method according to embodiment 62a, wherein the threshold value TV_{HLA-DR/CD62L(CD8+)N} is 0.3, 0.35, 0.4, 0.45, 0.48, 0.5, 0.6 or 0.7.

63c. The method according to embodiment 62b, wherein the threshold value TV_{HLA-DR/CD62L(CD4+)N} is between 0.2 and 0.8, such as between 0.3 and 0.7, such as between 0.4 and 0.6, such as between 0.45 and 0.5.

63d. The method according to embodiment 62b, wherein the threshold value TV_{HLA-DR/CD62L(CD4+)N} is 0.3, 0.35, 0.4, 0.45, 0.48, 0.5, 0.6 or 0.7.

63e. The method according to embodiment 62c, wherein the threshold value TV_{HLA-DR/CD62L(CD3+)N} is between 0.2 and 0.8, such as between 0.3 and 0.7, such as between 0.4 and 0.6, such as between 0.45 and 0.5.

63f. The method according to embodiment 62c, wherein the threshold value TV_{HLA-DR/CD62L(CD3+)N} is 0.3, 0.35, 0.4, 0.45, 0.48, 0.5, 0.6 or 0.7.

64. The method according to embodiment 62a, 62b, 62c, 63a, 63b, 63c, 63d, 63e or 63f wherein the samples S6 and S7 are blood.

65. The method according to any one of embodiments 62a, 62b, 62c, 63a, 63b, 63c, 63d, 63e, 63f or 64, wherein at least two of the samples S1, S2, S3, S4, S6 and S7 are the same sample.

66a. The method according to any one of the embodiments 1 to 45, wherein the method further comprises the steps of:
determining the proportion of CD8⁺T-cells which are HLA-DR⁺ in a sample S6 from the subject and/or determining the total number of CD8⁺ T-cells which are HLA-DR⁺ in a predetermined volume of a sample S6 from the subject;
determining the proportion of CD8⁺ T-cells which are CD62L⁺ in a sample S7 from the subject and/or determining the total number of CD8⁺ T-cells which are CD62L⁺ in a predetermined volume of a sample S7 from the subject;
calculating the ratio of the proportions of HLA-DR⁺ CD8⁺ T-cells and CD62L⁺ CD8⁺ T-cells and/or the ratio of the total numbers of HLA-DR⁺ CD8⁺ T-cells and CD62L⁺ CD8⁺ T-cells;
comparing said ratio with a threshold value TV_{HLA-DR/CD62L(CD8+)P} and/or TV_{HLA-DR/CD62L(CD8+)N}; and
wherein, if said subject suffers from inflammatory bowel disease, a ratio higher than said threshold value TV_{HLA-DR/CD62L(CD8+)P} and/or TV_{HLA-DR/CD62L(CD8+)N} is indicative of that said subject suffers from ulcerative colitis.

66b. The method according to any one of the embodiments 1 to 45, wherein the method further comprises the steps of:
determining the proportion of CD4⁺T-cells which are HLA-DR⁺ in a sample S6 from the subject and/or determining the total number of CD4⁺ T-cells which are HLA-DR⁺ in a predetermined volume of a sample S6 from the subject;
determining the proportion of CD4⁺ T-cells which are CD62L⁺ in a sample S7 from the subject and/or determining the total number of CD4⁺ T-cells which are CD62L⁺ in a predetermined volume of a sample S7 from the subject;
calculating the ratio of the proportions of HLA-DR⁺ CD4⁺ T-cells and CD62L⁺ CD4⁺ T-cells and/or the ratio of the total numbers of HLA-DR⁺ CD4⁺ T-cells and CD62L⁺ CD4⁺ T-cells;
comparing said ratio with a threshold value TV_{HLA-DR/CD62L(CD4+)P} and/or TV_{HLA-DR/CD62L(CD4+)N}; and
wherein, if said subject suffers from inflammatory bowel disease, a ratio higher than said threshold value TV_{HLA-DR/CD62L(CD4+)P} and/or TV_{HLA-DR/CD62L(CD4+)N} is indicative of that said subject suffers from ulcerative colitis.

66c. The method according to any one of the embodiments 1 to 45, wherein the method further comprises the steps of:
determining the proportion of CD3+T-cells which are HLA-DR⁺ in a sample S6 from the subject and/or determining the total number of CD3⁺ T-cells which are HLA-DR⁺ in a predetermined volume of a sample S6 from the subject;
determining the proportion of CD3⁺ T-cells which are CD62L⁺ in a sample S7 from the subject and/or determining the total number of CD3⁺ T-cells which are CD62L⁺ in a predetermined volume of a sample S7 from the subject;
calculating the ratio of the proportions of HLA-DR⁺ CD3⁺ T-cells and CD62L⁺ CD3⁺ T-cells and/or the ratio of the total numbers of HLA-DR⁺ CD3⁺ T-cells and CD62L⁺ CD3⁺ T-cells;
comparing said ratio with a threshold value TV_{HLA-DR/CD62L(CD3+)P} and/or TV_{HLA-DR/CD62L(CD3+)N}; and
wherein, if said subject suffers from inflammatory bowel disease, a ratio higher than said threshold value TV_{HLA-DR/CD62L(CD}3)P and/or TV_{HLA-DR/CD62L(CD3+)N} is indicative of that said subject suffers from ulcerative colitis.

67a. The method according to embodiment 66a, wherein the threshold values TV_{HLA-DR/CD62L(CD8+)P} and/or TV_{HLA-DR/CD62L(CD8+)N} are between 0.2 and 0.8, such as between 0.3 and 0.7, such as between 0.4 and 0.6, such as between 0.45 and 0.5.

67b. The method according to embodiment 66a, wherein the threshold values TV_{HLA-DR/CD62L(CD8+)P} and/or TV_{HLA-DR/CD62L(CD8+)N} are 0.3, 0.35, 0.4, 0.45, 0.48, 0.5, 0.6 or 0.7.

67c. The method according to embodiment 66b, wherein the threshold values TV_{HLA-DR/CD62L(CD4+)P} and/or TV_{HLA-DR/CD62L(CD4+)N} are between 0.2 and 0.8, such as between 0.3 and 0.7, such as between 0.4 and 0.6, such as between 0.45 and 0.5.

67d. The method according to embodiment 66b, wherein the threshold values TV_{HLA-DR/CD62L(CD4+)P} and/or TV_{HLA-DR/CD62L(CD4+)N} are 0.3, 0.35, 0.4, 0.45, 0.48, 0.5, 0.6 or 0.7.

67e. The method according to embodiment 66c, wherein the threshold values TV_{HLA-DR/CD62L(CD3+)P} and/or TV_{HLA-DR/CD62L(CD3+)N} are between 0.2 and 0.8, such as between 0.3 and 0.7, such as between 0.4 and 0.6, such as between 0.45 and 0.5.

67f. The method according to embodiment 66c, wherein the threshold values TV_{HLA-DR/CD62L(CD3+)P} and/or TV_{HLA-DR/CD62L(CD3+)N} are 0.3, 0.35, 0.4, 0.45, 0.48, 0.5, 0.6 or 0.7.

68. The method according to embodiment 66a, 66b, 66c, 67a, 67b, 67c, 67d, 67e or 67f wherein the samples S6 and S7 are blood.

69. The method according to any one of embodiments 66a, 66b, 66c, 67a, 67b, 67c, 67d, 67e, 67f or 68, wherein at least two of the samples S1, S2, S3, S4, S6 and S7 are the same sample.

70. The method according to any one of embodiments 1 to 69, wherein the samples S1, S2, S3, S4, S5, S6 and S7 are independently chosen from the group consisting of blood, plasma, serum, or faeces.

71. Method of treatment of Crohn's disease in a subject, wherein the method comprises performing the steps of a method for determining if a subject suffers from Crohn's disease or from ulcerative colitis according to any one of embodiments 1 to 70, and, when the subject is found to suffer from Crohn's disease, the method of treatment further comprising the step of administering to said subject at least one medicament for treating Crohn's disease.

72. The method of treatment according to embodiment 71, wherein the medicament is chosen from the group consisting of steroids, such as budesonide, prednisolone, and betamethasone, or immune modulator, such as azathioprine, mercaptopurine and methotrexate, or biological treatments such as, adalimumab and infliximab, or 5-aminosalicylates, such as sulfasalazine, mesalamine, balsalazide, and olsalazine, or antibiotics, such as metronidazole.

73. Method of treatment of ulcerative colitis in a subject, wherein the method comprises performing the steps of the method for determining if a subject suffers from Crohn's disease or from ulcerative colitis according to any one of embodiments 1 to 70, and, when the subject is found to suffer from ulcerative colitis, the method of treatment further comprising the step of administering to said subject at least one medicament for treating ulcerative colitis.

74. The method of treatment according to embodiment 73, wherein medicament may be chosen from the group consisting of steroids, such as prednisolone and betamethasone, or 5-aminosalicylates, such as sulfasalazine, mesalamine, balsalazide, and olsalazine, or biological treatments such as, adalimumab and infliximab, or calcineurin inhibitors, such as cyclosporine, tacrolimus and sirolimus, or immune modulators, such as azatioprine and mercaptopurine.

1. A method for determining if a subject suffers from Crohn's disease or from ulcerative colitis, wherein the method comprises the steps of:
determining if the subject suffers from inflammatory bowel disease;
determining the proportion of B-cells which are CD27⁺ in a sample S1 from the subject;
comparing said proportion with a threshold value TV_{CD27}; and
wherein, if said subject suffers from inflammatory bowel disease, a proportion lower than said threshold value TV_{CD27} is indicative of that said subject suffers from Crohn's disease; and
wherein, if said subject suffers from inflammatory bowel disease, a proportion higher than said threshold value TV_{CD27} is indicative of that said subject suffers from ulcerative colitis.

2. The method according to claim 1, wherein the threshold value TV_{CD27} is between 5% and 30%.

3. The method according to claim 1, wherein the method further comprises the step of
determining the proportion of B-cells which are CD23⁺ in a sample S2 from the subject;
calculating the ratio of the proportions of CD23⁺ B-cells and CD27⁺ B-cells;
comparing said ratio with a threshold value TV_{CD23/CD27}; and
wherein, if said subject suffers from inflammatory bowel disease, a proportion higher than said threshold value TV_{CD23/CD27} is indicative of that said subject suffers from Crohn's disease; and
wherein, if said subject suffers from inflammatory bowel disease, a proportion lower than said threshold value TV_{CD23/CD27} is indicative of that said subject suffers from ulcerative colitis.

4. The method according to claim 3, wherein the threshold value TV_{CD23/CD27} is between 0.5 and 8.

5. The method according to claim 1, wherein each of the samples S1 and S2 independently is a blood sample or a tissue sample.

6. The method according to claim 1, wherein the step of determining that the subject suffers from inflammatory bowel disease comprises:
determining the amount of hemoglobin in a sample S3 and the amount of orosomucoid in a sample S4 from the subject;
calculating the ratio of the amount of hemoglobin in the sample S3 and the amount of orosomucoid in the sample S4;
comparing said ratio with a threshold value TV_{H/O};
wherein, a ratio lower than said threshold value TV_{H/O} is indicative of that said subject suffers from inflammatory bowel disease.

7. The method according to claim 6, wherein the threshold value TV_{H/O} is between 100 and 200.

8. The method according to claim 1, wherein the step of determining that the subject suffers from inflammatory bowel disease comprises:
determining the amount of hemoglobin in a sample S3 and calprotectin in a sample S5 from the subject;
calculating the ratio of the amounts of hemoglobin in the sample S3 and calprotectin in the sample S5;
comparing said ratio with a threshold value TV_{H/C};
wherein, a ratio lower than said threshold value TV_{H/C} is indicative of that said subject suffers from inflammatory bowel disease.

9. The method according to claim 8, wherein the threshold value TV_{H/C} is between 0.1 and 5.

10. The method according to claim 1, wherein the step of determining that the subject suffers from inflammatory bowel disease comprises the steps of:
determining the amount of orosomucoid in a sample S4 from the subject;
comparing said amount of orosomucoid with a threshold value for orosomucoid TV_{O};
wherein, an amount of orosomucoid higher than said threshold value TV_{O} is indicative of that said subject suffers from inflammatory bowel disease.

11. The method according to claim 10, wherein the threshold value TV_{O} is between 0.5 and 1.0 g/L blood.

12. The method according to claim 1, wherein the step of determining that the subject suffers from inflammatory bowel disease comprises the steps of:
determining the amount of calprotectin in a sample S5 from the subject;
comparing said amount of calprotectin with a threshold value TV_{C} for calprotectin;
wherein, an amount of calprotectin higher than said threshold value TV_{C} is indicative of that said subject suffers from inflammatory bowel disease.

13. The method according to claim 12, wherein the threshold value TV_{C} is between 50 and 300 mg/kg faeces.

14. The method according to claim 1, wherein the method further comprises the steps of:
determining the proportion of CD8⁺ T-cells which are HLA-DR⁺ in a sample S6 from the subject;
comparing said proportion with a threshold value TV_{HLA-DR};
wherein, a proportion of CD8⁺ T-cells which are HLA-DR⁺ higher than said threshold value TV_{HLA-DR} is indicative of that said subject suffers from ulcerative colitis.

15. The method according to claim 14, wherein the threshold value TV_{HLA-DR} for the proportion of CD8⁺ T-cells which are HLA-DR⁺ is between 10 and 40%.

16. The method according to claim 1, wherein the method further comprises the steps of:
determining the proportion of CD3⁺ T-cells which are HLA-DR⁺ in a sample S6 from the subject;
comparing said proportion with a threshold value TV_{HLA-DR} for CD3⁺ T cells;
wherein, a proportion of CD3⁺ T-cells which are HLA-DR⁺ higher than said threshold value TV_{HLA-DR} for the proportion of CD3⁺ T-cells which are HLA-DR⁺ is indicative of that said subject suffers from ulcerative colitis.

17. The method according to claim 1, wherein the samples S1, S2, S3, S4, S5 and S6 are independently chosen from the group consisting of blood, plasma, serum, faeces, or tissue sample.

18. CD27⁺ B-cells for use as a marker in the diagnosis of Crohn's disease.

19. The ratio of CD23⁺ B-cells and CD27⁺ B-cells for use as markers in the diagnosis of Crohn's disease.

20. The ratio of hemoglobin and orosomucoid for use as a marker in the diagnosis of inflammatory bowel disease.

21. The proportion of CD8⁺-T-cells which are HLA-DR⁺ for use as a marker in the diagnosis of ulcerative colitis.

22. The proportion of CD3⁺T-cells which are HLA-DR⁺ for use as a marker in the diagnosis of ulcerative colitis.

23. Kit for the diagnosis of Crohn's disease and/or ulcerative colitis comprising:
a B-cell marker
antibodies directed against CD27; and
instructions for use.

24. A kit according to claim 23 further comprising:
antibodies directed against CD23.

25. The kit according to claim 23 further comprising:
a T-cell marker
antibodies directed against HLA-DR; and
optionally antibodies directed against CD62L.

26. The kit according to any one of claims 23 further comprising:
reagents to measure levels of orosomucoid.
